(19) United States
(12) Patent Application Publication
(10) Pub. No.: US 2006/0177922 A1
(43) Pub. Date: Aug. 10, 2006

(54) Environmental control incubator with removable drawer and robot

(75) Inventors: Benjamin Nathan Shamah, Palo Alto, CA (US); Eric James Rollins, Sonora, CA (US); Reuben Sandler, Berkeley, CA (US); Nilesh Chhaganlal Mistry, Hayward, CA (US); David K. Matsumoto, San Jose, CA (US); Ryan Powell, Menlo Park, CA (US); Thomas Lawrence Smith, Rodeo, CA (US); JoeBen Bevirt, Santa Cruz, CA (US); Russell T. Berman, San Francisco, CA (US); Ian Yates, Menlo Park, CA (US)

Correspondence Address: Fernandez & Associates, LLP, PO Box D, Menlo Park, CA, 94025-6204, US

(73) Assignee: Velocity 11, Menlo Park, CA (US)

(21) Appl. No.: 11/055,899

(22) Filed: Feb. 10, 2005

(51) Int. Cl. C12M 1/34; C12M 1/36

(52) U.S. Cl. 435/286.2; 435/303.1; 435/287.3

(57) ABSTRACT

An incubator for storing micro-plates or micro-tubes comprises a handling robot positioned between shelves or drawers containing micro-titer plates or other containers useful for biological based reactions. The advantages of this configuration are the ultimate compactness of the system and increased speed or reliability achieved with more than one robot being able to access the same plate or tube. Alternative embodiments standardize the spacing and configuration of a robot track and a shelf track such that a shelf and a robot are interchangeable in a track.

### FIELD OF INVENTION

Invention relates to an environmentally controlled chamber which promotes biologically based reactions on multiple plates under predetermined conditions with robotic placement and retrieval of the reaction plates.

### BACKGROUND OF INVENTION

Cabinets of special construction for biological process investigation first appeared in the 1920's as microbiological incubators manufactured by the forerunner of Heraeus Instruments. Today incubators are used to store plates for a certain time at prescribed environmental conditions. In cell-based assay protocol, media and cells are added to empty plates that are then placed in the incubator to grow overnight. Typical environmental specs are 37° C. at 95% relative humidity with a 5% CO₂ environment. During the following day plates are removed to add assay material and then replaced, being removed again later that day for reading. Temperature stability requirements depend on throughput but are typically about ±1° C. Stability of the CO₂ supply at 5% during the run also depends on throughput and is about ±1%. Physical stability is also important as plate disturbances can disrupt cell growth. For chemical assays, components are added to empty plates and the plates are placed in the incubator at 37° C. and are incubated for some time, depending on the nature of the experiment. Plates come out, material is added, and the plates go back in. Later the plates are removed and read in a reader. Exact temperature stability requirements depend on throughput; in general about ±1° C. is required. For PCR amplification components are added to empty plates and the plates are placed in an incubator at 4-25° C. and are incubated for some time, depending on the nature of the experiment. Plates are then extracted, assayed, and returned to an incubator for storage. For PCR assays, temperature stability is not an important factor. Usage of an incubator for compounds of interest storage requires a generally stable environment but not a tightly controlled one. In such an application, allowing temperatures ranging from 4-25° C. for stored plates is acceptable. In an integrated system where environmental control is not an issue at all, an incubator or similar device may be used just for large volume plate handling and possibly for plate input and output. Current incubators hold about 250 plates; future units will require 1,000 plates or more.

Available incubators have plate storage density ranging from 2 to 9 plates per 1,000 in³; at 10 plates per 1,000 in³, 1,000 plates occupies the volume of about 64 cubic feet. Incubators often include some of the climate control modules within the machine and therefore offer a less plate-dense package than dedicated storage devices. Plate access times vary widely between manufacturers and models. Stated specifications are not always meaningful because the moves to which time values apply are not always clear. Real values could vary widely depending on whether the value refers to access time for a plate in the closest location or the farthest location in the storage chamber. Even if they are specified and accurate for access time, a measure of cycle time (time to replace a plate at the system access position with another plate in the same position) might be more meaningful. Better yet, the number of plates accessible per unit of time might be the most meaningful measure as it most closely represents the probable usage of the device. Typical stated access times are between 20 and 40 second with some manufacturers offering higher speed upgrades to faster access times (as low as 12 seconds). Reliability is the major problem that plagues this market, especially with top-loading incubators. The details of the specific requirements for an incubator differ between customers and according to each protocol. The varied protocols place a variety of demands on incubators with customers needing temperature control, humidity control, gaseous environments and particle filtration in multiple combinations and ranges.

Recently Liconic AG began manufacturing an “automatic storage device and climate controlled cabinet”, as detailed in U.S. 2004/0213651. Predecessors to this apparatus can be found in U.S. Pat. No. 5,735,587, U.S. Pat. No. 6,129,428, U.S. Pat. No. 6,478,524, U.S. 2004/0115101 and EP 1,443,101, all sharing a common inventor. Alternative concepts and inventions can be found in U.S. 2004/0212285, U.S. 2004/0207303, U.S. 2004/0152188 and U.S. Pat. No. 6,568,770.

The central carousel design of the Liconic cabinets hinders scaling to a larger number of racks; it also suffers from a productivity limitation in that only one robot can be engaged in the circular configuration of the racks, plates and robot. The rack and pinion drive of the Liconic robot mechanism has difficulty placing micro-titer plates in a compactly designed plate holder rack due to its more complicated resolution limitations requiring additional gear boxes. Additionally, (651) fails to teach how “automatic operation” is achieved without the use of positional sensors. The other inventions disclosed suffer from comparable deficiencies; one example is lack of positional knowledge of a micro-titer plate when in motion, compromising the apparatus' ability to move quickly and with minimum motions to its destination; one solution of this problem in the prior art is the requirement to return to a home position prior to completing an instruction. For instance, U.S. 2004/0152188 has not the ability to turn its micro-plate transport device in an angular motion; additionally it uses a chain drive, not conducive to vibration free motion or precise positioning. These apparatus are insufficient for today's needs of high throughput screening of massive numbers of samples as required in combinatorial protocols for biological assays or microbiological incubations. Accordingly, there is need for an environmentally controlled cabinet with rapid deployment and retrieval of micro-titer reaction plates which can be scaled to a large number of plates with improved compactness and productivity.

### SUMMARY OF INVENTION

Invention resides in the unique combination of a handling robot positioned between two rows of racks containing micro-titer plates or other containers useful for biological reactions. The advantages of this configuration are the ultimate compactness of the system with the invented incubator consuming unused volume in the lower half of a larger apparatus such as the Velocity11 BioCel®, the Thermo MultiScan Ascent, RTS Thurnall and others and the direct delivery of plates to positions within reach of the main robot, requiring no additional plate transfer step. Alternative embodiments standardize the spacing and configuration of a robot track and a shelf track such that a shelf and a robot are interchangeable in a track.

The invented incubator comprises an integrated environmental control unit (ECU) that delivers a stable environment of a predetermined gas composition, temperature and humidity protocol to a chamber with removable shelves containing racks that hold industry standard micro-titer plates. Alternative embodiments provide a controlled source of HEPA filtered gas, including ambient air or compositions containing predetermined mixtures of O₂, CO₂, N₂ and others; alternatively programmable humidity selection is provided. An integrated, yet modular ECU, allows relocation to different positions for different product embodiments serving different applications. By minimizing plate access time, especially as it affects door-open time, and providing a robust climate control system, the invented incubator provides stable and reliable control over a broad range of potential protocols for the user.

The invented incubator further comprises a robot with servo motors with position encoding technology; computer programmable electronics enables simultaneous control of motion in multiple axes, ensuring reliable actuator operation and time-optimized robot trajectories, enabling quick access to plates with the least possible physical disturbance during plate transfer. Barcode reading components and processes combined with plate orientation sensing allow for real-time process verification. More complete characterization of error modes through improved, extended, and in some cases, redundant sensing enables superior fault handling.

In one embodiment the invented incubator is a subsystem of a larger robotic system such as a Velocity11 BioCel®, in this instance software is architected to provide for a primary system to issue commands and control an incubator subsystem, enabling greater flexibility for the customer. In one embodiment, firmware is written in C with an industry standard ActiveX communication protocol, exposing only the highest-level functions required to operate the device to the user. The software architecture combined with extensive data collection and inventory mapping enable fast and efficient plate management. More intelligent motion profiles and steps reduce process times for initializing and avoid superfluous moves such as reinitializing after a door has been opened or moving to park positions unnecessarily between steps, deficiencies of the prior art.

In one embodiment the invented incubator is one or more incubators in communication with and coupled to a robotic system such as a Velocity11 BioCel® or others. This embodiment is meant to accommodate large numbers of micro-plates in what is termed a library; for instance, 1,000 or more plates may be stored in accessible positions. The current reliability of robotic mechanisms is less than desirable; one embodiment is configured such that each plate may be accessed by more than one robot. In one embodiment shelves and robots utilize a common track configuration such that their widths are identical and the means for mounting and registration on a floor track is identical enabling interchangeability of positions in an incubator.

### BRIEF DESCRIPTION OF DRAWINGS

FIG. 1 is a scale drawing of a single incubator cabinet with shelf open and robot extended.

FIGS. 2A and 2B are scale drawings of two incubator cabinets positioned symmetrically.

FIG. 3 is a detail drawing of an incubator shelf in the open position.

FIG. 4 is a drawing of an incubator bottom panel showing construction details.

FIG. 5 is a detail drawing of an incubator drawing showing various features.

FIG. 6 is a detail drawing of an incubator rack.

FIGS. 7A and 7B are detail drawings of an incubator robot configuration.

FIGS. 8A and 8B are front and side detail drawings of an incubator robot.

FIG. 9 is a detail drawing of a drive architecture used on an incubator robot.

FIGS. 10 A, B, C and D are detail drawings of a drive configuration for x, z and theta motions.

FIGS. 11A and B are detail drawings of a drive configuration for y motion.

FIGS. 12A and B are detail drawings of a drive configuration for z external motion.

FIGS. 13A and B are detail drawings of a drive configuration for z internal motion.

FIGS. 14A and B are detail drawings of a drive configuration for theta motion.

FIG. 15 is a detail drawing of a drive configuration for x motion.

FIG. 16 is a system schematic showing various electronic control systems.

FIG. 17 is a schematic diagram showing how an incubator processor may access various databases and library files.

FIG. 18 is an incubator system schematic showing how a user may set mechanical adjustments for each axis and other control parameters.

FIG. 19 is a flow schematic showing how the robot and shovel is brought to a “home” position.

FIG. 20 is a flow schematic showing how micro-plates may be inventoried.

FIGS. 21A through F are flow schematics showing how micro-plates may be moved.

FIG. 22 is a flow schematic showing how a robot may be taught a position.

FIG. 23 is a schematic drawing of a three track incubator enclosure module.

FIGS. 24A and 24B are schematic drawings of a shelf for use in a multi-track incubator enclosure module.

FIG. 25 is a schematic drawing of a robot for use in a multi-track incubator enclosure module.

FIG. 26 is a schematic drawing of a three track module with two robots and one shelf.

FIG. 27 is a schematic drawing of a three track module with one robot and two shelves.

FIGS. 28A and 28B are isometric schematic drawings of an incubator library comprising three track incubator enclosure modules, shelves, robots and end covers.

FIG. 29 is a top view schematic drawing of a library comprising three track incubator modules, shelves, robots and end covers wherein there is one robot per two shelves.

FIG. 30 is a top view schematic drawing of an incubator library comprising three track modules, shelves, robots and end covers wherein there are two robots per one shelf.

FIG. 31 is a top view schematic drawing of an incubator library comprising three track modules, shelves, robots and end covers wherein the shelves and robots have been separated for maintenance access.

FIG. 32 is a top view schematic drawing of 8 incubator libraries comprising three track modules, shelves, robots and end covers wherein there is one robot per two shelves and a common conveyance.

FIG. 33 is an isometric view schematic drawing of 2 incubator libraries comprising three track modules, shelves, robots and end covers wherein there is one robot per two shelves and a common conveyance.

FIG. 34 is an isometric view schematic drawing of an incubator library comprising one three track module, two shelves, one robot and end covers wherein the robot and shelves slide out the narrow end door.

FIGS. 35A, B and C are isometric, top and alternative schematic views of an incubator library comprising four three track incubator modules, four shelves, five robots, three environmental control modules plus control systems, access door and common conveyance in communication with another robotic system.

FIGS. 36A and B are schematic views of a micro-plate access door in an end wall of a multi-track environmental control incubator unit.

FIGS. 37A and B are isometric views of an alternative micro-plate access door in an end wall of a multi-track environmental control incubator unit.

### DETAILED DESCRIPTION

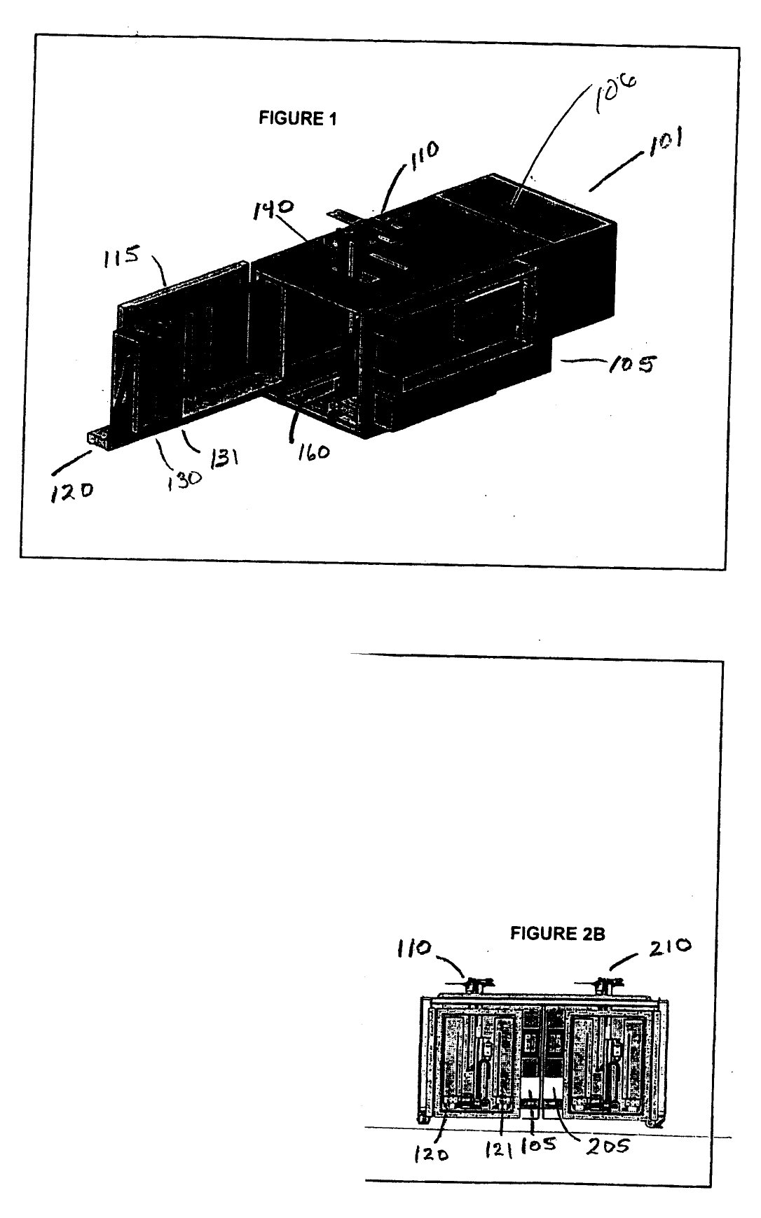

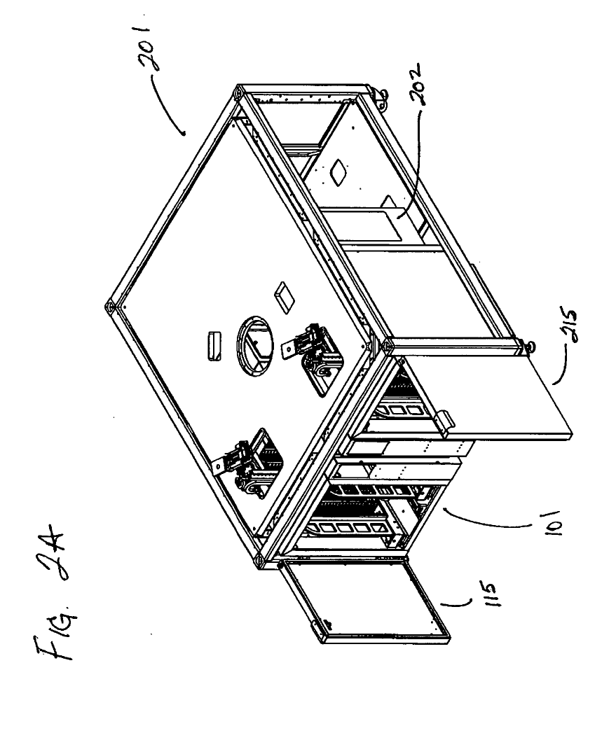

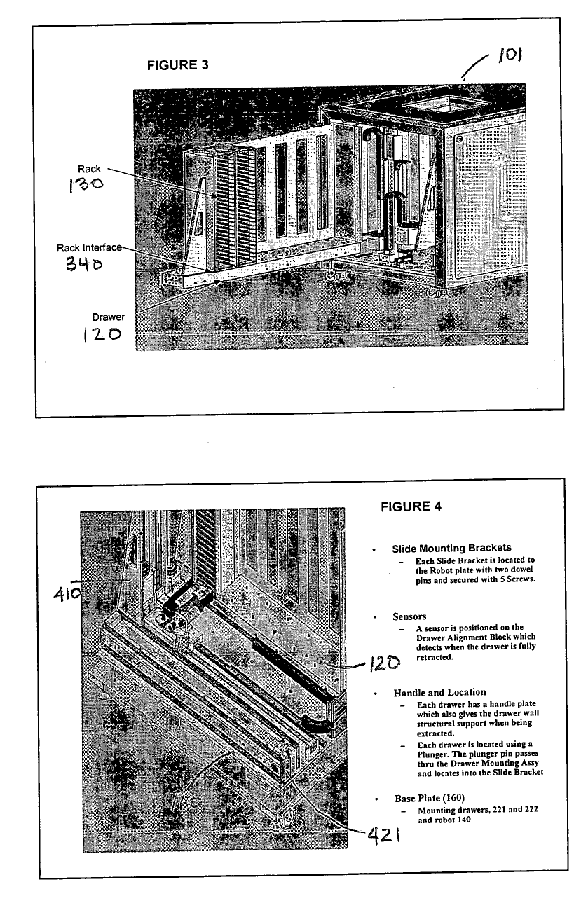

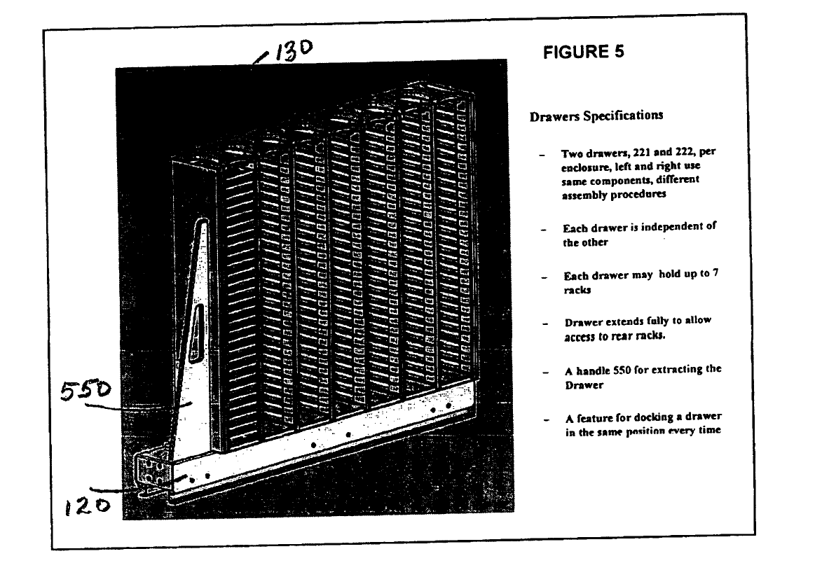

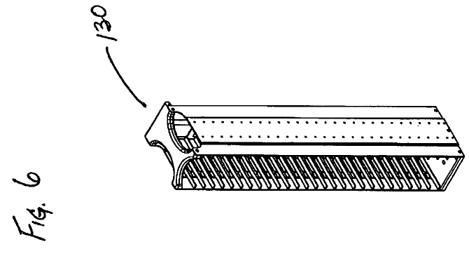

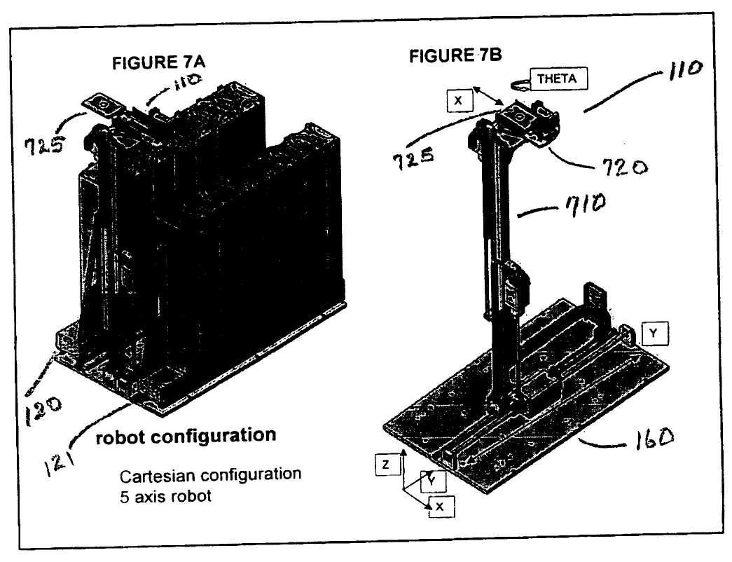

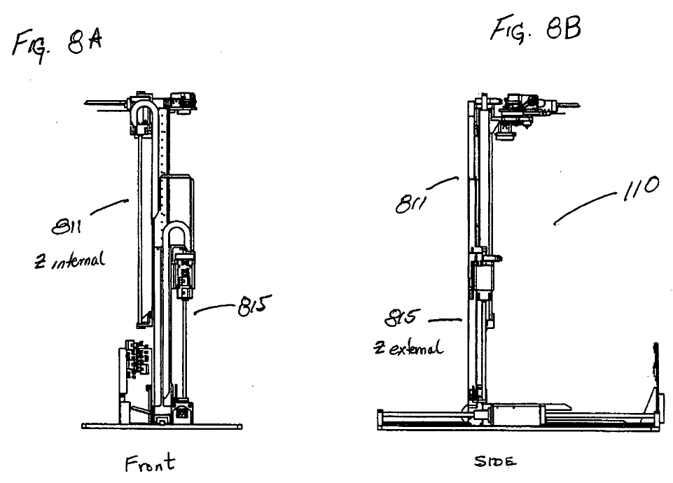

FIG. 1 shows incubator 101, electronics enclosure 105, extended robot 110, door 115, sliding shelf 120 and plate racks 130 and 131, second door 140, open, and base plate 160 to which two shelves, 120 and 121 (not shown) and five axis robot 110 are attached. FIGS. 2A and 2B are perspective and front views of two incubators 101 and 202 mounted in a mirror image arrangement 201, showing electronics 105 and 205 mounted on the interior surface, extended robots 110 and 210, doors 115 and 215. FIG. 3 is a higher detail drawing of incubator 101 with shelf 120 in the open position showing rack 130 with rack interface 340. FIG. 4 is a drawing of an incubator bottom portion showing construction details of two shelves 120 and 421 and lower robot detail 410. FIG. 5 is a detail drawing of an incubator shelf 120 showing space for up to seven racks such as 130. A handle 550 is configured as a support element for the shelf and racks. FIG. 6 is a detail drawing of an incubator rack 130 showing slots for 28 micro-titer plates or other substrates; vents to allow airflow over the plates are in the backside, not shown, of the rack. Racks in an invented incubator can be loaded either using a manipulator with plates supplied by a larger major system, such as from a BioCel® robot, or by the user opening a door and placing a rack in manually. Plate presence sensing is used also to confirm that a plate has been properly retracted by the shovel. Another goal of plate presence sensing is to determine whether there is a plate in the rack so that a five axis robot does not try to deliver a plate into a full shelf. Plate presence sensing can also be used during the inventory function in conjunction with a barcode reader to confirm plate presence for the database. The barcode reader is used to confirm the plate identity before each pick as well as to perform a plate inventory. An inventory is performed by scanning all of the plates in the racks and reporting their identity to the database without removing the plates from the racks. Plate orientation sensing is accomplished by sensing corner keys with LED's and reflective spots in one embodiment. Racks may have a barcode placed on the base of the rack to be used as an identifier; in one embodiment a rack barcode contains additional information such as shelf spacing so that multiple plate types can be stored in an incubator. Alternatively an RFID identification chip, LED chip or reflective spot may be placed on a rack and/or micro-titer plates to facilitate plate presence or orientation or rack location or identification.

FIGS. 7A and 7B are detail drawings of an incubator robot 110 configuration. FIG. 7A shows robot 110 positioned between two rows of racks 130 with its arm 710 fully extended. The top portion 720 swivels at least 180° and extends plate holder shovel portion 725 in the x direction. As shown in FIGS. 8A and 8B arm 710 is divided into at least two portions, 811 termed Z internal or Z1 and 815 termed Z external or Z2. Z external is attached to Z internal; Z internal operates within the confines of incubator 101; Z external, with portion 720 attached to it, may extend outside of the cabinet to place and retrieve plates or substrates from exterior positions. Note from FIGS. 7 and 8 that robot 110 may travel in the y direction the length of an incubator 101, has motion ability in the x direction to access each level of a rack, may move in the z direction up and down within the incubator via motion mechanism Z internal, extend out of the incubator with motion mechanism Z external and turn about 280° with theta motion mechanism 725 which is part of portion 720. When top portion 720 is outside of the enclosure the theta motion may increase to about 360°. Robot 110 is termed a five-axis robot operating in a Cartesian coordinate system; the five axes being, X, Y, Z1, Z2 and theta.

The invented incubator delivers plates through a programmable door 140 in the top of its enclosure to the BioCel®, in one embodiment, or any other integrated system or itself to positions within a selected angular (yaw) and vertical range. This range encompasses at least four possible positions: two robot accessible landscape orientations (south and north) at two distinct heights separated by a minimum distance equal to the height of the tallest possible plate or other consumable substrate plus overhead. An incubator is modularly connectable and in communication with a BioCel® or other major robotic system such that it returns to approximately the same place from which it was removed when reconnected. The repeatability of this positioning is improved by a teaching process, designed to be as simple as possible. In one embodiment, three internal components of an incubator are two rack shelves, 120 and 121, and a five axes robot, 110; each of these is mounted on a base plate, 160. A base plate may be removed from a cabinet of an incubator with these components attached; this maintains the positional orientation of these components. Cleaning an incubator enclosure and major components is facilitated by being able to remove internal components. Antiseptic cleaning of all internally exposed surfaces is a key factor in preventing cross-contamination of plate cultured experiments.

It is critical to some protocols that door 140 to the external environment be open a minimum amount of time in order to reduce perturbations to the internal environment of the incubator; calculating or sensing robot 110 position as it approaches door 140 in order to minimize door open time is a key feature of invented incubator in some embodiments. Alternatively, a load-lock transfer station may be placed above door 140 such that a means of matching the atmosphere and pressure of the location the plate is being transferred to or from is enabled.

The environmental control unit (ECU) enables a programmable set of environmental variables comprising predetermined gas compositions, temperature and humidity protocols, virtually microbial-free HEPA filtered gas, including ambient air or compositions containing mixtures of O₂, CO₂, N₂ and others. At least one sensor for, optionally, measuring temperature, moisture, gas composition, air velocity, plate vibration, internal cabinet pressure, electromagnetic radiation level and particle count; in alternative embodiments a time stamped record is kept of all sensor readings in a processor accessible library. One or more fans are located in appropriate points in the enclosure to facilitate circulation. In one embodiment the cabinet is hermetic and may be operated at pressures above or below atmospheric except when transferring a plate in or out; alternatively a load-lock transfer station may be placed above the door such that the load-lock station provides a means of matching the atmosphere and pressure of the location the plate is being transferred to or from. The humidity control may be achieved by providing a source of sterile water and a means to flow a gas through the water, such as a bubbler; the flow through the bubbler is based on the humidity desired and that sensed; alternatively a commercially available moisture delivery system may be incorporated into the ECU portion. Optionally, a HEPA filter may be included in the ECU enclosure to reduce particles in the air or fluid stream internal to an incubator. Alternatively, chemical adsorbent filters may be added in situations where the internal gas composition is controlled. Other alternatives include a cabinet with a mechanical pump or other means which enables pressure control, either less or greater than atmospheric. The construction method and material of the incubator cabinet is determined by the requirements for sterility, hermeticity, radiation protection and internal pressure.

Including an environmental control system, an incubator follows a modular design path, allowing ready access to all components, including the ability to detach and service as individual components. Additionally, it possible to remove any rack manually (and thus any plate) when the machine is not functioning properly or is not powered. Restart procedures are fault-tolerant and facilitate returning the machine back on line without data loss. In extreme cases, conversational language bypassing ActiveX controls may be used to operate an incubator.

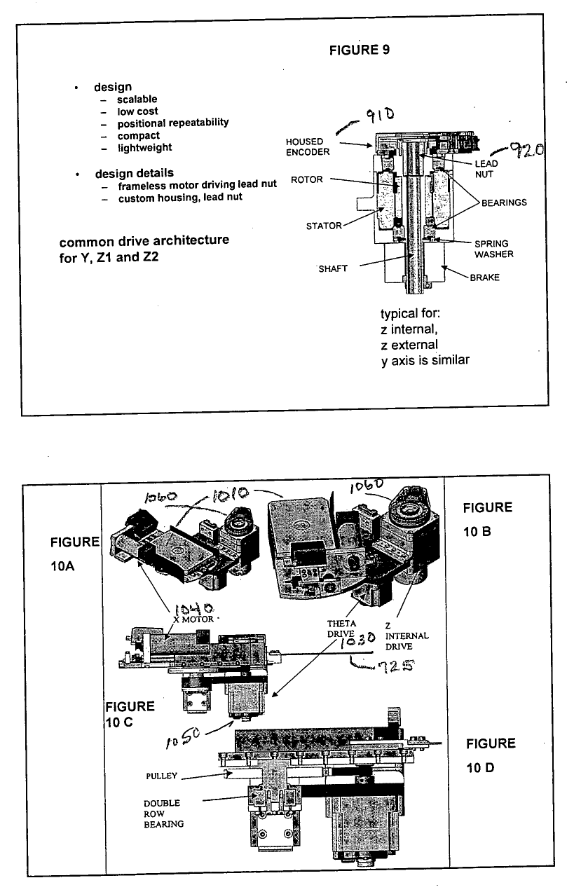

The Cartesian layout of the invented incubator is superior to the prior art due to scalability, the ability to maximize the usable space under the BioCel® (rectangular vs. square layout). General consideration was given to factors of: environment survivability, scalability, reliability, speed, elegance, innovation, and cost. All motions are generated from a rotational motor, shown in FIGS. 9 and 10. The drive assembly performance is defined by:

distance of travel

maximum acceleration

maximum velocity

positional repeatability

maximum load

In one embodiment a robot is configured so that it mounts to the same base plate, 160, as shelves, 120 and 121. This gives reference datum for the robot motion to be aligned with the planes of the shelves and then to the racks in order to maintain repeatability of plate positions. The linear motions of y, z internal, z external, and x are all guided by linear rails of decreasing size. The z internal motion covers the entire height of the chamber allowing a single axis to cover all plate locations (in the z axis). The z external motion is used only to extend out from the chamber in order to reach the plate pads on the deck of a BioCel®, or other major system or the incubator itself. Base plate, 160, contains all electrical and service connections for operation of a five axis robot; such connections are engaged upon sliding a base plate into an incubator enclosure.

The structural integrity of a robot is built up from the base plate through the joints connecting linear bearings up to the theta axis. The highest loads are applied to the joint between the y axis and the z external column. The situation causing the highest force is a maximum acceleration or deceleration move by the y axis when z internal is at the maximum height. Movement of the y axis when z external is at the maximum height position is disallowed in most embodiments.

The y, z internal, and z external are designed with a common architecture. Each of the three axes has a motor, frameless or not, which drives a lead nut on a lead screw fixed at both ends. This configuration is compact and scalable to long lengths (limited by the sag of the lead screw). The compactness comes from the ability to nest the lead nut and bearing supports for each drive axis. Housed differential encoders are use to provide positional feedback for each of the axes. FIG. 9 is a detail drawing of a drive architecture used on the incubator robot for the z internal, z external and y motion. Note that motion motors for the Z1, Z2 and Y axes share a common drive architecture. Housed encoder 910 and lead nut 920 are key components in sensing the position of plate, 1010, and plate holder shovel, 725 shown in FIG. 10. Positional sensors, as encoders 1060, on theta drive, 1030, and X, 1040, drive motor assemblies provide additional data for accurately locating and recording the position of plate holder shovel 725. The position resolution of each axis drive is determined by the pitch of the threads on the lead screw and lead nut; sensing the rotations of the lead nut of a particular axis drive provides the data for calculation of the position of a plate holder shovel at any point in time. This positional data is acquired by sensors and may be processed by an internal incubator processor and/or communicated to an external major, or master, system for processing and decision making. In one embodiment a “home” position is designated by a “home sensor” and “homing flag” on every axis; in a start-up procedure, a homing routine is executed for the robot to learn where it is. An optical encoder, resolver and potentiometer are provided on each axis; 1050 is the location of these items for the theta drive.

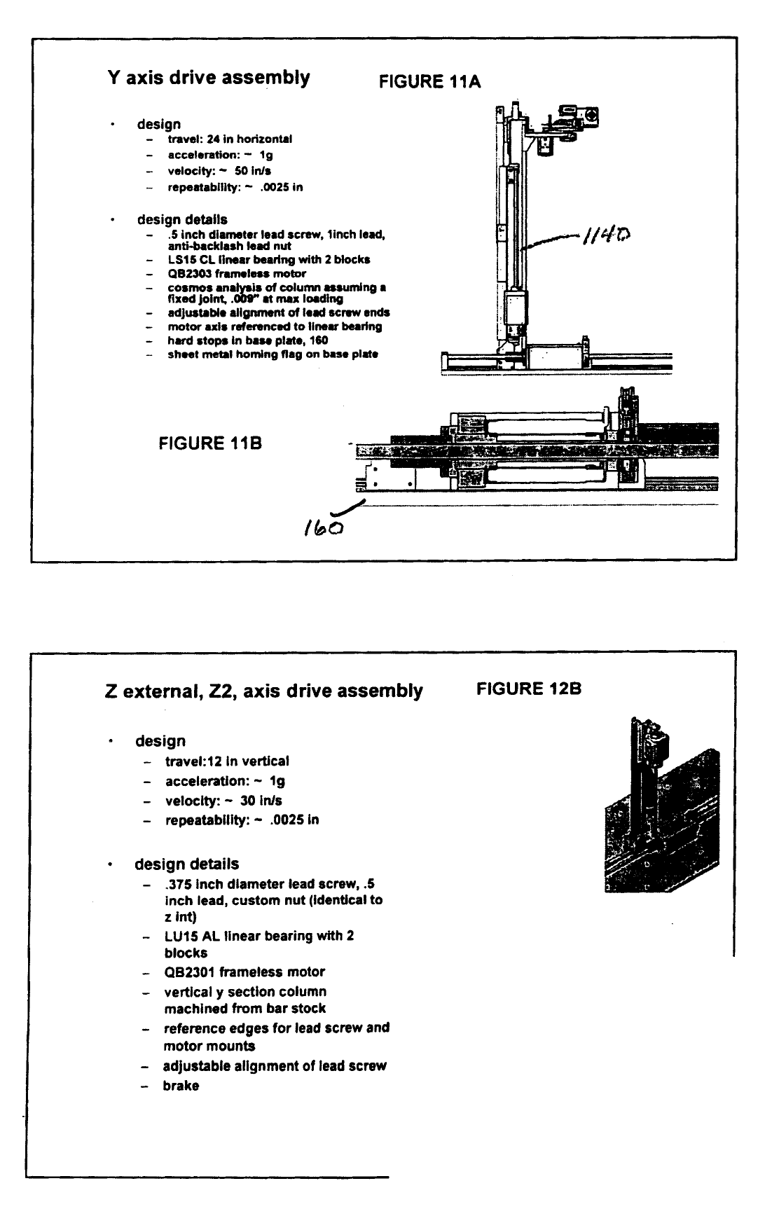

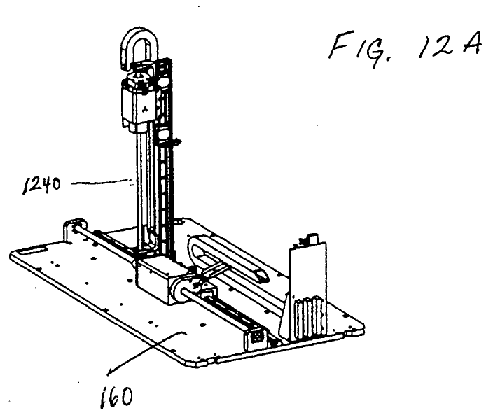

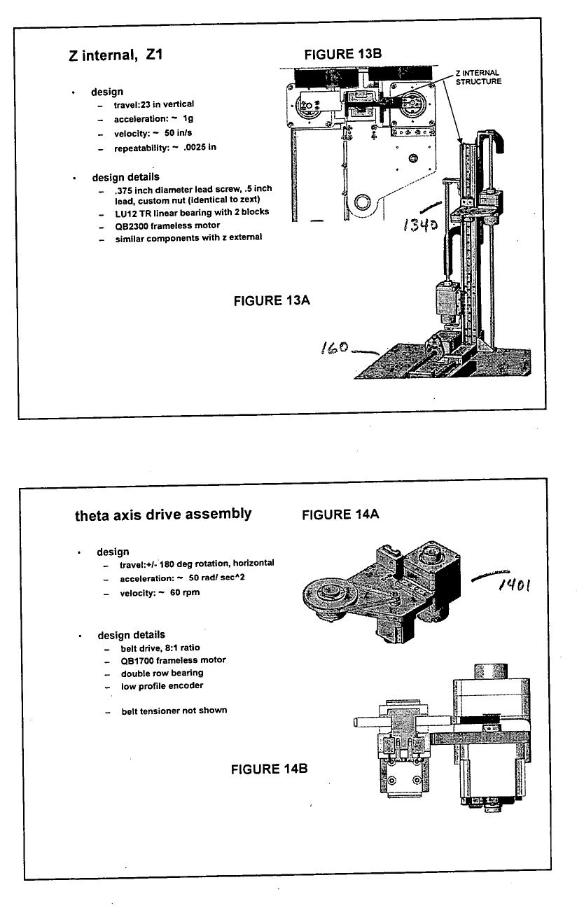

FIGS. 11A and B are detail drawings of a drive configuration for Y motion, showing lead screw 1140. In one embodiment a Y axis extends approximately 24 inches; alternative embodiments extend this dimension to at least 48 inches; the motor and positional sensing means are capable of a positional placement repeatability of at least ±0.0025 inches; alternative embodiments with a finer pitch lead screw and nut improve this repeatability to at least ±0.0010 inches. FIGS. 12A and B are detail drawings of a drive configuration for Z external, Z2, motion showing lead screw 1240. In one embodiment a Z2 axis extends approximately 12 inches; alternative embodiments extend this dimension to at least 24 inches the motor and positional sensing means are capable of a positional placement repeatability of at least ±0.0025 inches; alternative embodiments with a finer pitch lead screw and nut improve this repeatability to at least ±0.0010 inches. FIGS. 13A and B are detail drawings of a drive configuration for Z internal, Z1 motion showing lead screw 1340. In one embodiment a Z1 axis extends approximately 24 inches; alternative embodiments extend this dimension to at least 48 inches; the motor and positional sensing means are capable of a positional placement repeatability of at least ±0.0025 inches; alternative embodiments with a finer pitch lead screw and nut improve this repeatability to at least ±0.0010 inches.

The theta axis rotates the x axis, shovel and plate assembly, in order to access both rows of racks within an incubator as well as to reach a range of drop off positions at a table top. Drive assembly 1401 is a size 17 motor, frameless or not, which uses a belt drive to rotate the shaft of the x axis. Rotational accuracy is maintained by maximizing the gear reduction and encoder count on the motor. FIGS. 14A and B are detail drawings of a drive configuration for theta motion. In one embodiment the belt drive has a 8:1 ratio and can rotate ±180° in a horizontal plane; a belt tensioner is not shown. Alternative embodiments have a different reduction ratios and can rotate more than 180° in a horizontal plane. As one knowledgeable in the art will understand different types of motors, including frameless or not, may be used depending on the user requirements.

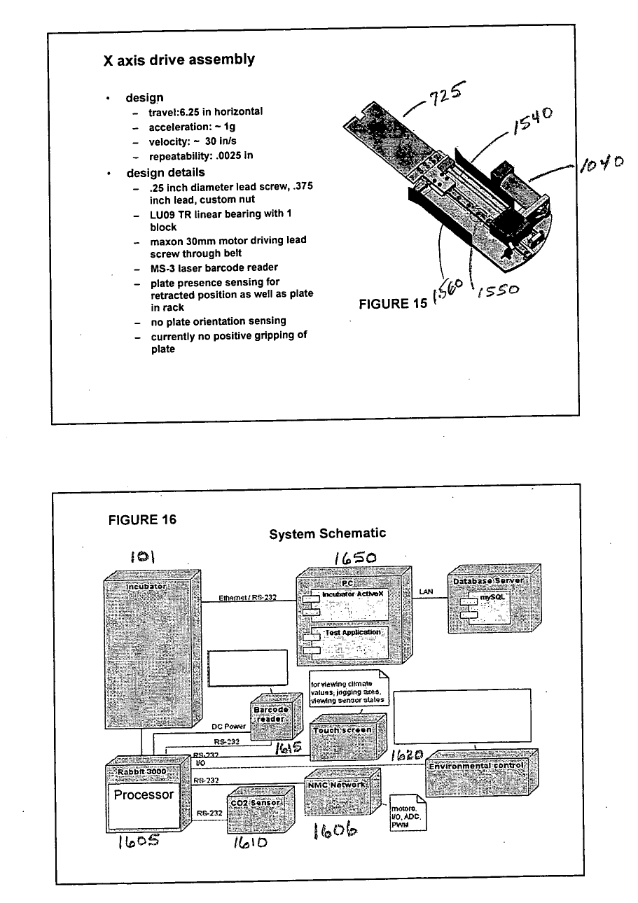

The function of the x axis drive assembly 1501, shown in FIG. 15, is to extend a plate holder shovel, 725, which must pick up a plate, such as 1010. A shovel is extended below a plate and then a vertical motion allows the plate to nest in the shovel before it is retracted. The advantage of a shovel gripper design is the low overhead of physical space which allows increased density of plate storage. However, with such a low overhead it becomes more difficult to ensure that all plates can be handled securely at high speeds. This shovel design also requires less accuracy in the y direction for plate handling. The guides on the shovel base realign and re-center the plate during each retraction of a shovel. The x axis drive assembly is designed as a spinning lead screw, 1540, attached to the carriage. The carriage moves on a linear bearing, 1550, to support and guide the motion. The motor spins the lead screw through a belt drive. FIG. 10 A, B, C and D are detail drawings of a drive configuration for x, z and theta motions. In one embodiment, not shown in FIG. 10 or 15, a camera is positioned above the plate holder shovel such that it may be inserted into the rack and observe the status of various wells in a micro-titer plate of interest; alternatively the camera may be a photodetector capable of sensing radiation of energies of interest such as ultra-violet or infra-red. Alternatively, the camera may be positioned such that it does not enter the rack but observes the plate from an external position. In one embodiment means to sense the position of each rack and each shelf is incorporated into X axis drive assembly 1501. In one embodiment plate handling is done with a shovel mechanism assisted by a gripper during high speed moves.

Other embodiments that were considered include a spinning lead screw. This option was ruled out due to whip of a long lead screw. Belts were explored but have the problem of the required tension of a belt over a long distance of travel. The structure needed to support the required tension would increase the mass, driving up the required torque over such a long distance, approximately 36 inches. In order to maintain repeatability, higher cost linear encoders would be required. The primary advantage of the belt system is that motors could be fixed at the base, which has advantages for cabling.

FIG. 16 is a system schematic showing various electronic control systems. In one embodiment all of these items are located within electronics enclosure 105 as part of incubator 101. Sensor 1610 is labeled as a CO₂ sensor in this embodiment; in alternative embodiments one or more sensors may be located throughout an incubator 101 measuring parameters of interest such as temperature, humidity, gas composition, air velocity, vibration, internal cabinet pressure, electromagnetic radiation level and particle count; in alternative embodiments a time stamped record is kept of all sensor readings. Alternatively the computing 1605 and 1650 and networking 1606 capability may be located exterior to incubator 101; for example a BioCel® or equivalent may contain all or a portion of the computational and file storage needs of incubator 101. At a minimum communications capability via Ethernet or RS232 or equivalent standard is retained internal to an invented incubator. In one embodiment, a user interface is a touch screen, 1620, which may be mounted in an accessible location on an incubator to provide a user with direct monitoring of the internal incubator state and provides some local control over parameters, if appropriate, within the context of the selected protocol and the status of the various sensors and other monitoring devices. Software enables tightly monitored access to plates in an incubator during runs and records changes to ensure data and process integrity. The software program also maintains past movement logs to facilitate error recovery.

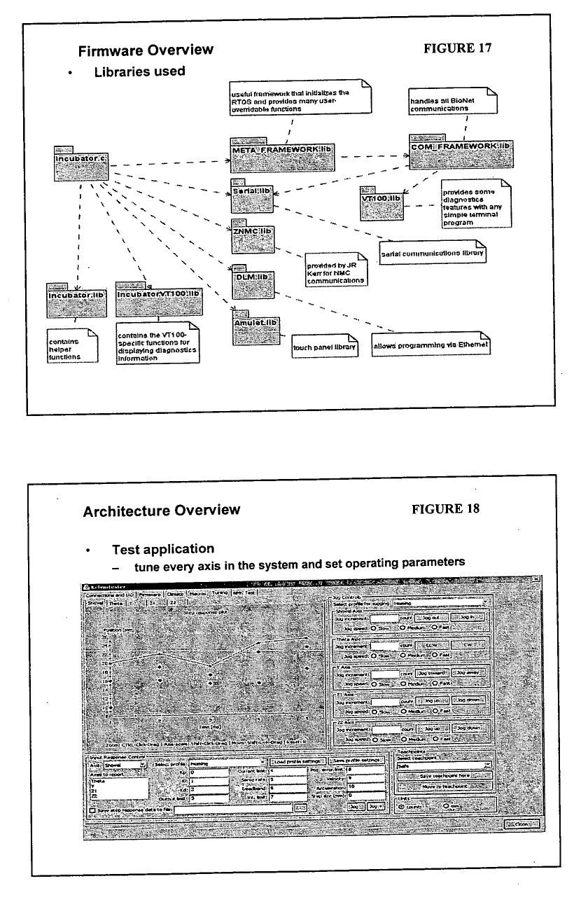

FIG. 17 is one example of how incubator 101 and CPU 1605 access various internal databases and library files; alternatively some or all of these files may be located external to incubator 101, for instance in a BioCel®. Communication is via alternative interfaces such as serial or Ethernet. Electric power requirements are optionally 110 or 220 VAC. The incubator manipulator is connected to the integrated system emergency stop circuit to avoid unsafe conditions. Dedicated software allows seamless integration into larger systems, for instance a BioCel®, and present an intuitive user interface, 1620, requiring a minimum of setup and training. A simple and repeatable docking procedure allows users or service personnel to disengage an incubator from a BioCel® or other integrated system, move it away for service or system reconfiguration, and then replace and realign it quickly.

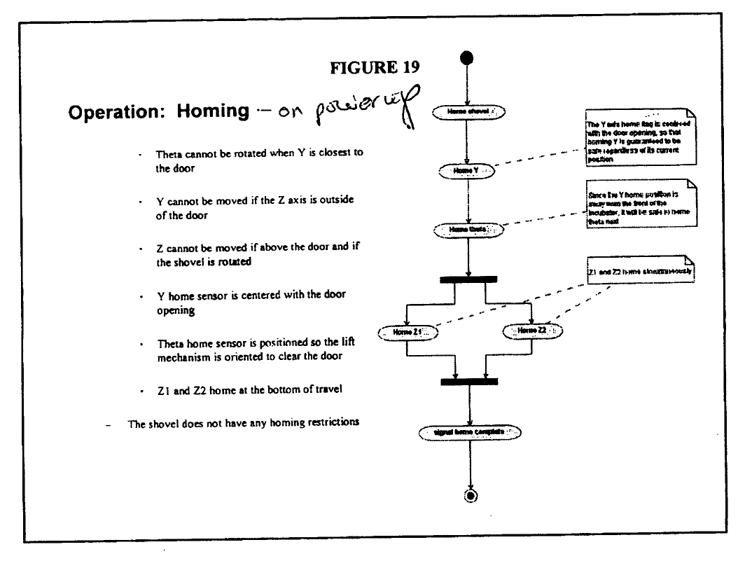

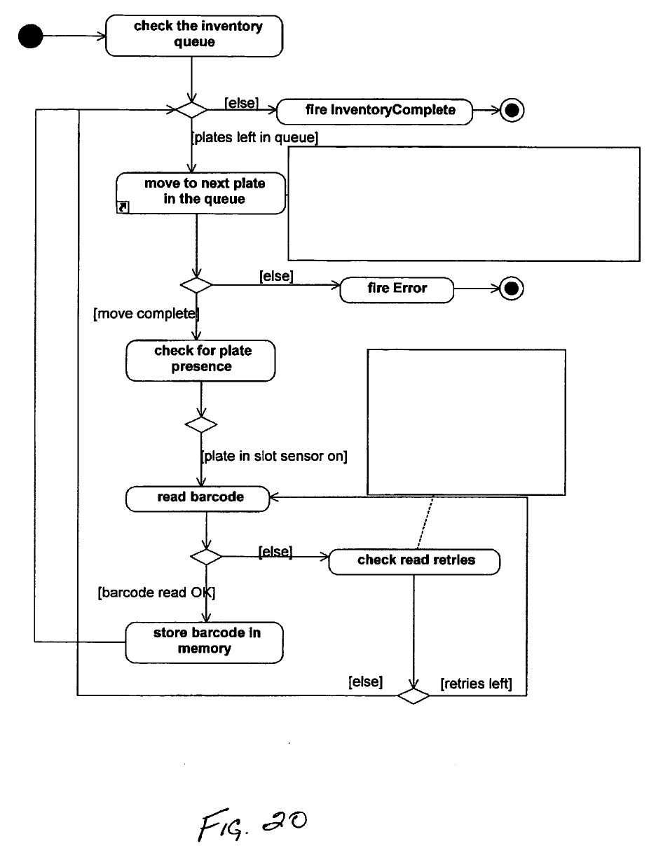

FIG. 18 is a system schematic showing how a user may set mechanical adjustments for each axis and other control parameters. Each axis uses optical sensors to provide a homing signal. The homing routine is designed to prevent a collision for all axes from any position. FIG. 19 is a flow schematic showing how the robot and shovel is brought to a “home” position. Various safeguards are programmed into instruction software to avoid collisions. In one embodiment the door 140 retracts as robot 110 approaches it based on an instruction from processor 1605 or external processor, in a BioCel® for instance. The position of robot 110 may be calculated by data supplied by the optical sensors on each axis; alternatively the information may be supplied by a bar code reader positioned below door 140 [not shown] which also contains, for instance, a LED sensor for detecting robot 110.

FIG. 20 is a flow schematic showing how plates may be inventoried.

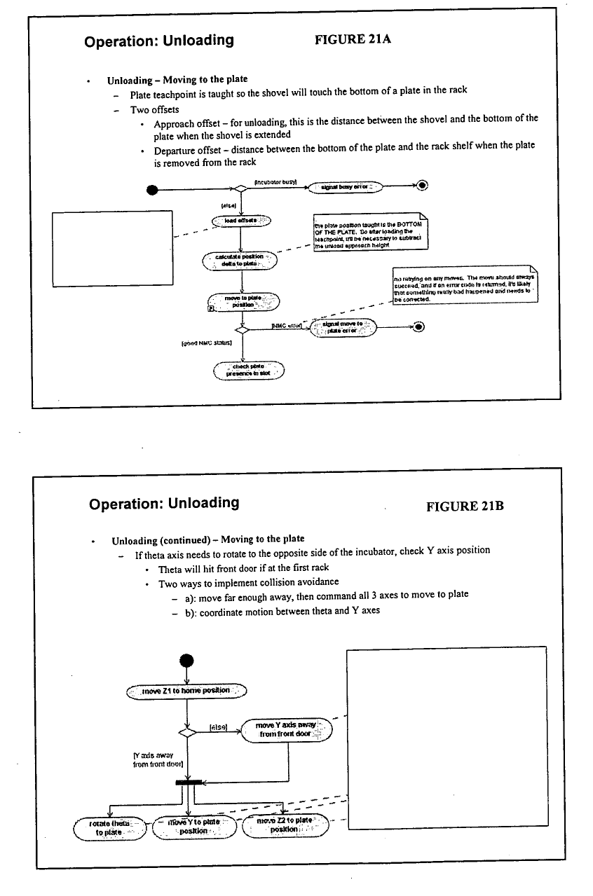

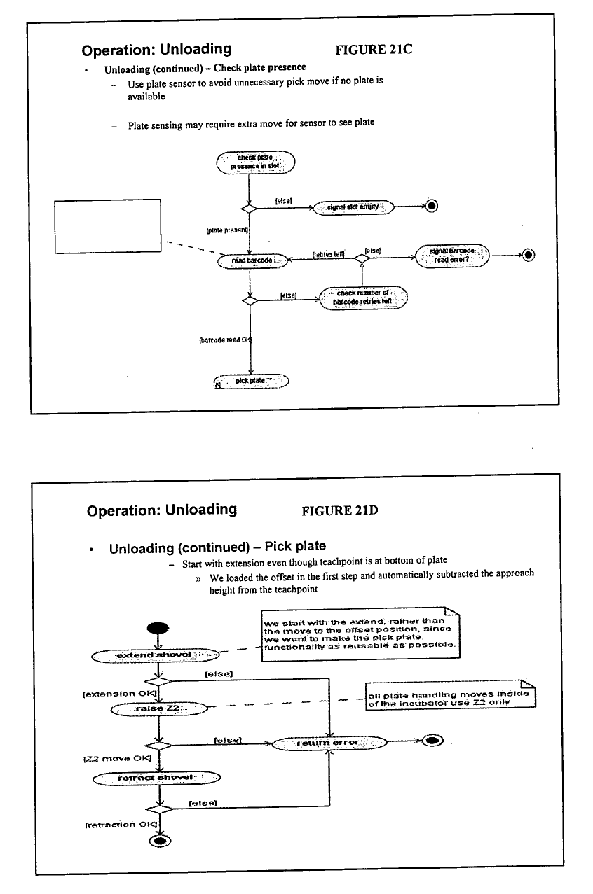

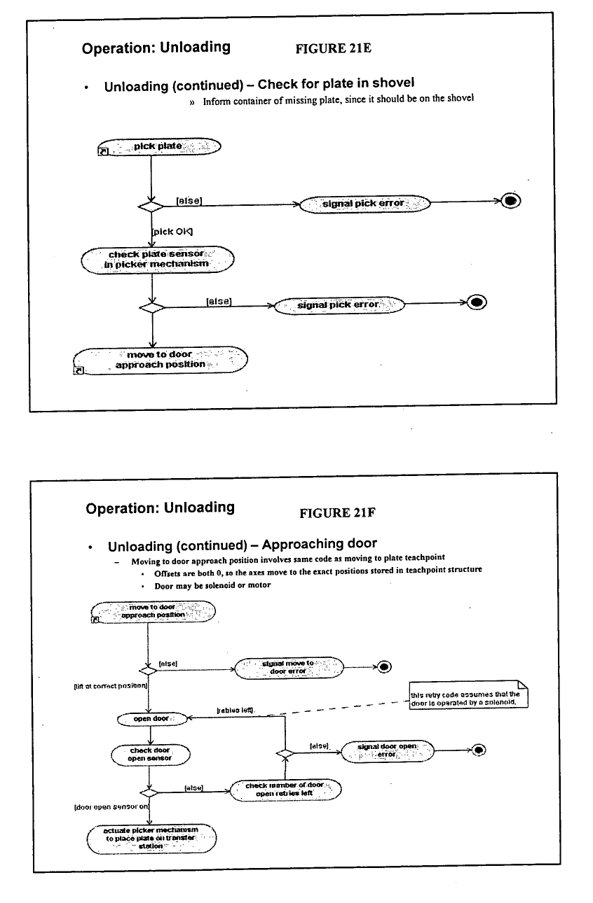

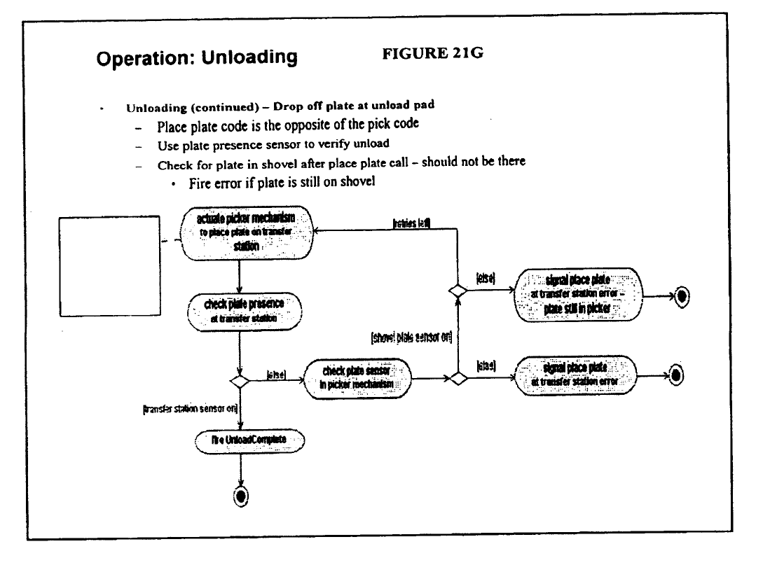

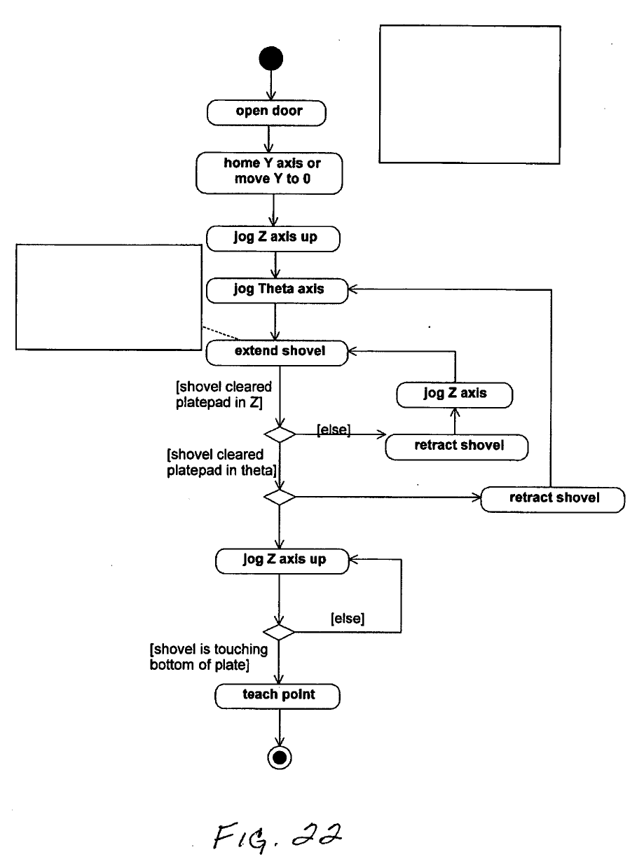

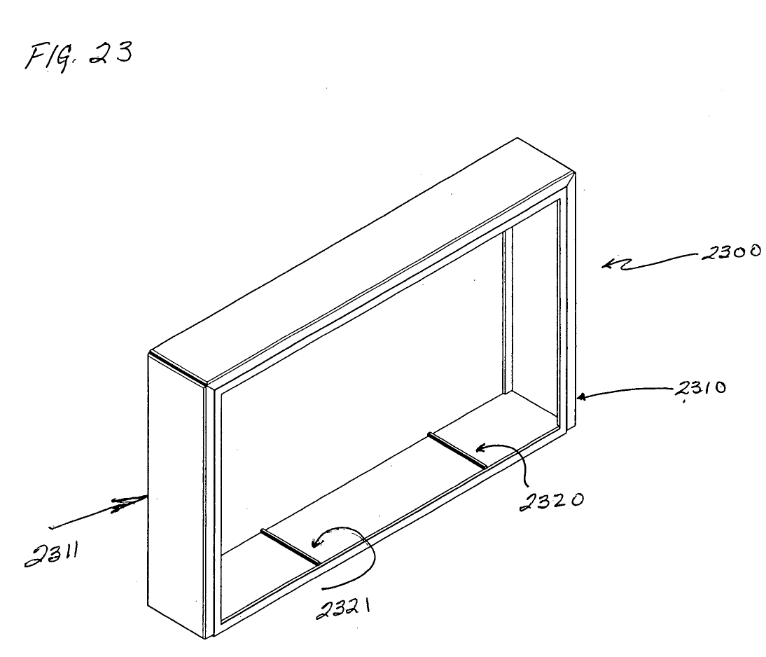

FIG. 21A through F are flow schematics showing how plates may be moved. The general sequence to deliver a plate from inside the chamber to a BioCel® or other instrument plate pad in one embodiment is:

Receive plate identification from processor 1605 or external command;

Determine position of robot 110;

Move robot 110 along y to the specified rack centerline; simultaneously

Move robot 110 along z internal to the approach position of the specified plate shelf; simultaneously

Rotate robot 110 along theta to the correct angle (0 deg for racks 1 thru 7, 180 deg for racks 8 thru 16);

Detect plate with barcode reader or corner key sense;

Extend shovel 725 along X axis to predetermined amount;

Move robot 110 along z internal up above the plate shelf by predetermined amount;

Retract shovel 725 along X axis by predetermined amount;

Move robot 110 along y, theta, z internal, toward the top door approach position;

Open top door 140 as directed by processor 1605;

Verify door open;

Move robot 110 along z external to deck plate pad approach height;

Move robot 110 along theta and extend shovel to plate pad;

Move robot 110 along z external down to place plate;

Retract shovel 725;

Move robot 110 along theta to top door approach position;

Retract robot 110 along z external to bottom position;

Close top door 140.

Communicate with robotic system such as the Velocity11 BioCel®

FIG. 22 is a flow schematic showing how the robot may be taught a position.

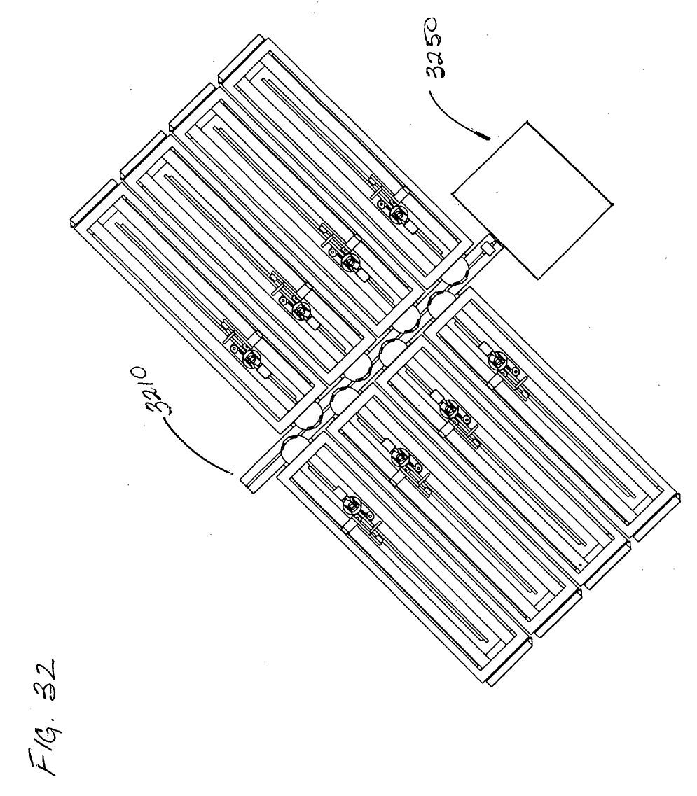

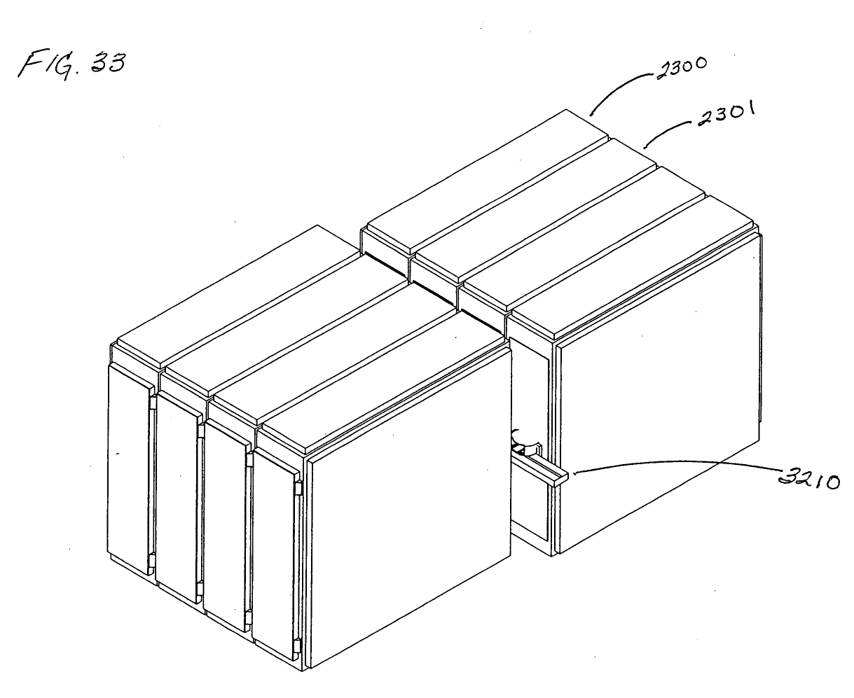

In applications where large numbers of micro-plates or micro-tubes are employed different embodiments may require one or more incubators coupled together. In one embodiment a modular incubator comprises a modular enclosure 2300 shown in FIG. 23 capable of containing one or more shelves, serving the function of a drawer in embodiments for smaller numbers of micro-plates, and one or more robots; optionally the module is sized for combinations of three or four tracks, each track holding a shelf or robot, interchangeably. In the embodiment shown in FIG. 23 end walls 2810 and 2811, shown in FIG. 28, optionally may be removable for access to shelves or robots; optionally narrow end walls 2310 and 2311 may contain environmental control apparatus, computer processing means, air and electrical ducting, a large access door and other means to facilitate the operation of a modular incubator, and at least one end wall contains one or more plate access doors, not shown. Cross-tracks 2320 and 2321 provide for placing and adjusting the position of shelves and robots in the “X” direction, that direction perpendicular to the long direction of enclosure 2300.

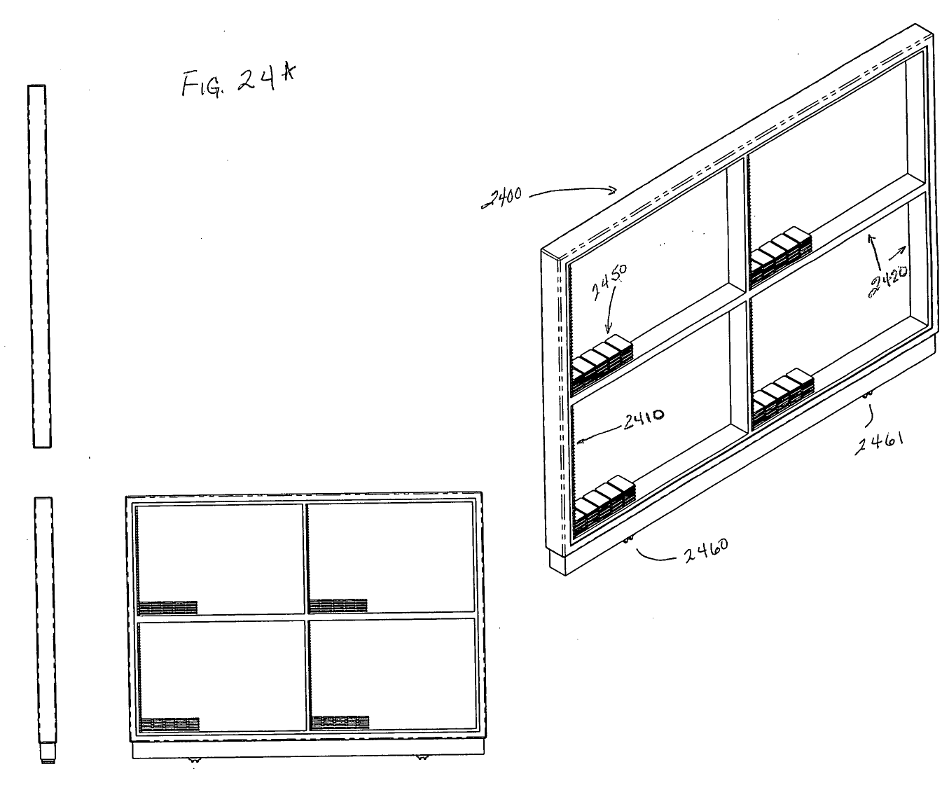

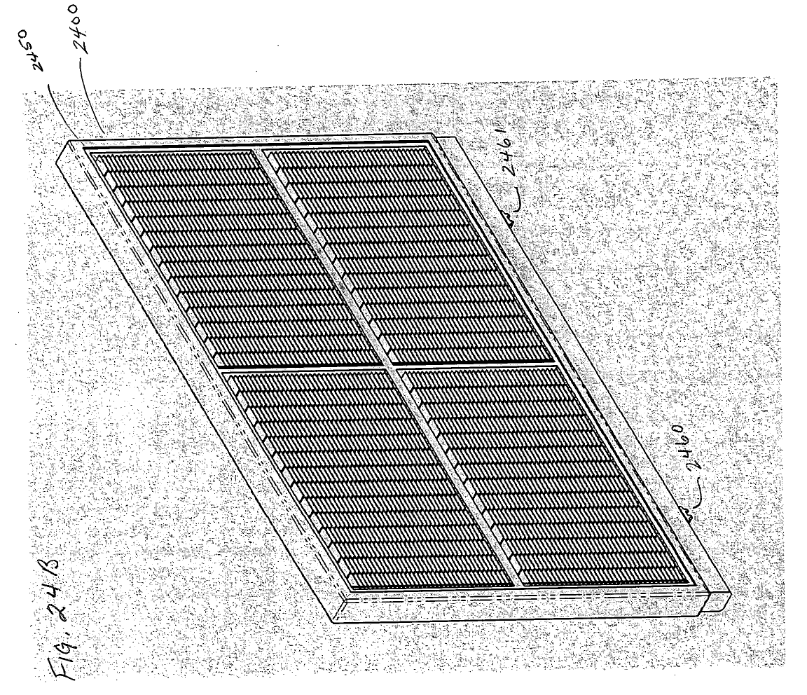

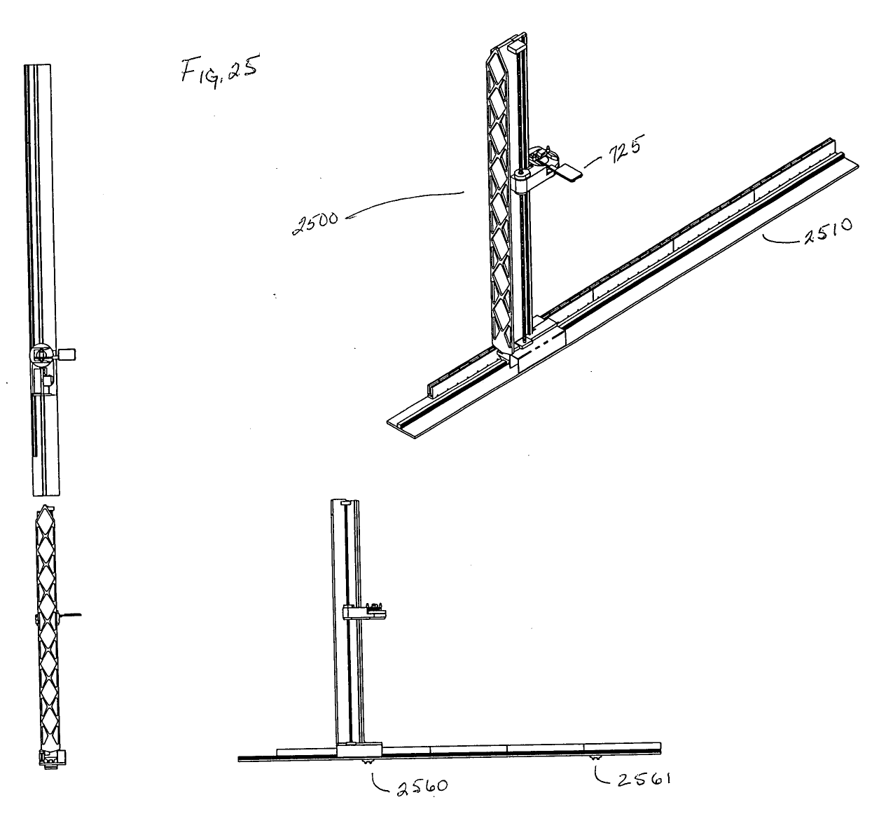

A shelf 2400, as shown in FIG. 24A, comprises a plurality of plate support tabs 2410, not all shown, and support beams 2420; each micro-titer plate 2450 is supported by two plate support tabs. In one embodiment a shelf may be about 8 feet long by about 8 feet tall by about 6 inches wide; a robot track may be somewhat longer but identical in width. FIG. 24B shows a fully loaded shelf. FIG. 25 shows robot 2500 and associated track 2510 for traversing in the “Y” direction. Note slides 2460 and 2461 for a shelf and 2560 and 2561 for a robot which engage cross tracks 2320 and 2321 of enclosure 2300, permitting motion in the “X” direction. Shelves and robots are placed into or removed from enclosure 2300 in an order desired with the aid of cross-tracks 2320 and 2321.

In one embodiment robot 2500 has at least four axes, for instance, x, y, z and theta. A plate gripper may be added to work in concert with the plate holder shovel, 725. Primary tasks of a robot in an environmental incubator are to retrieve a specific plate from a shelf upon command, deliver that plate to a plate access door, not shown, for transfer to another conveyance means, accept a plate from another conveyance means through a plate access door and deliver a plate to a given shelf plate holder position as directed.

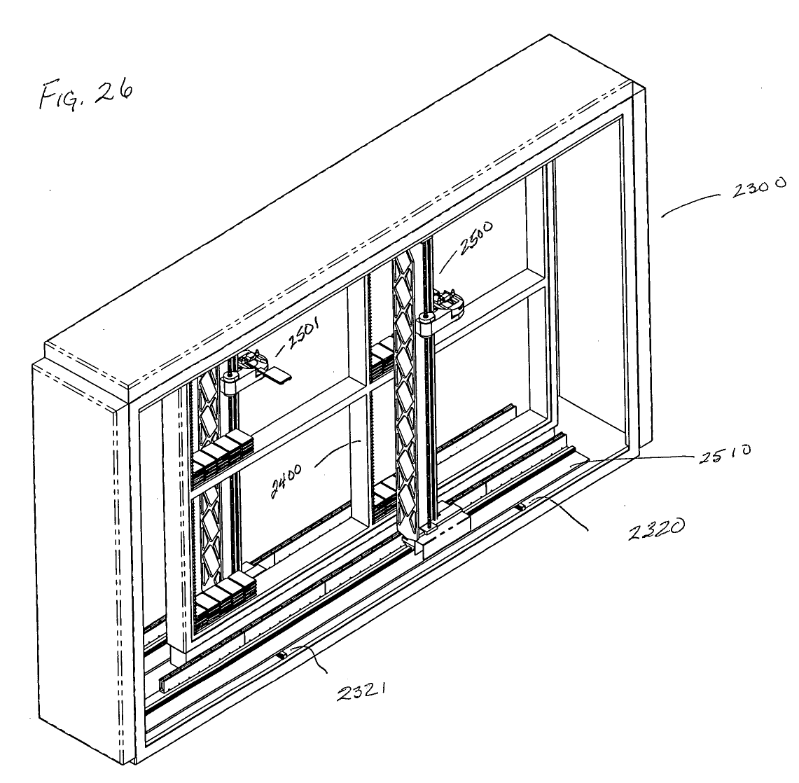

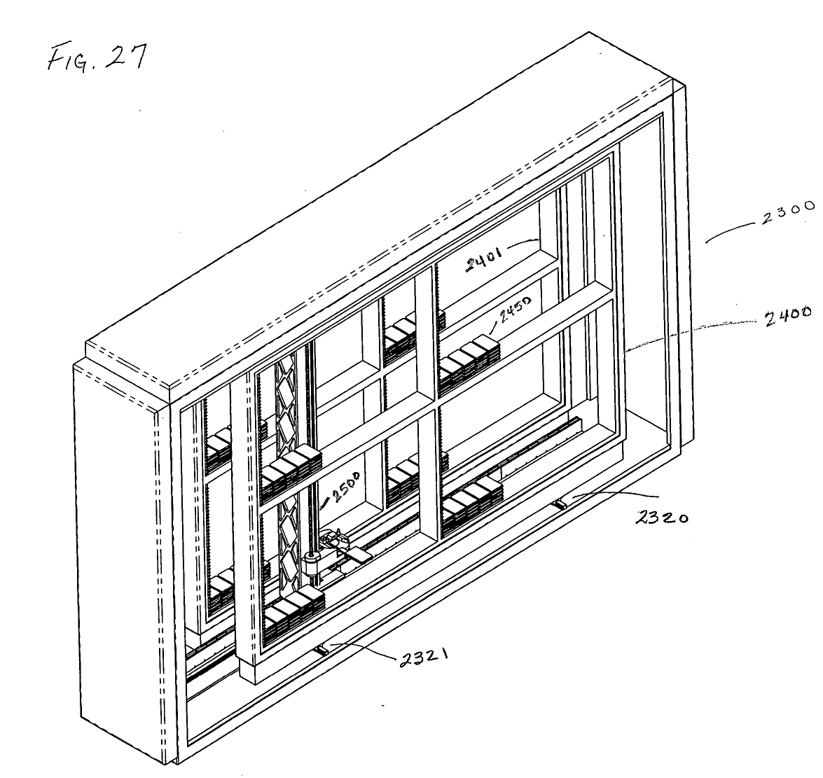

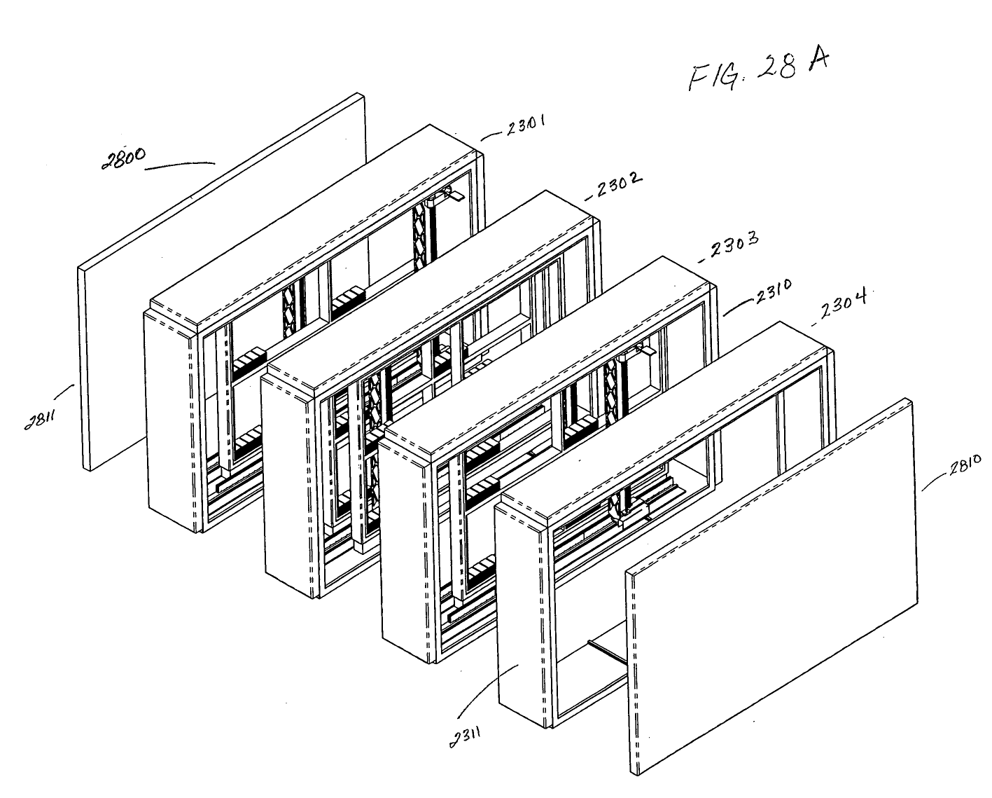

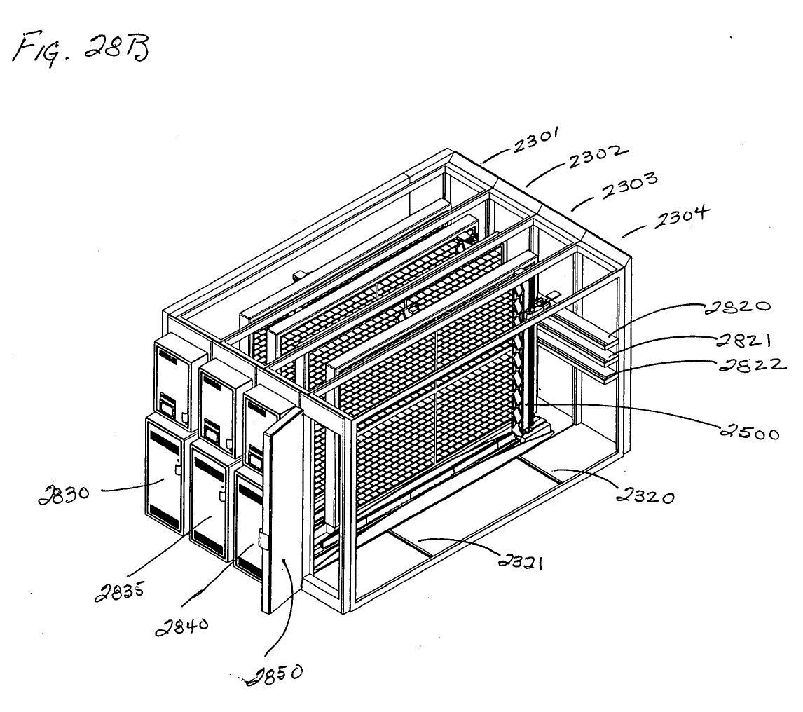

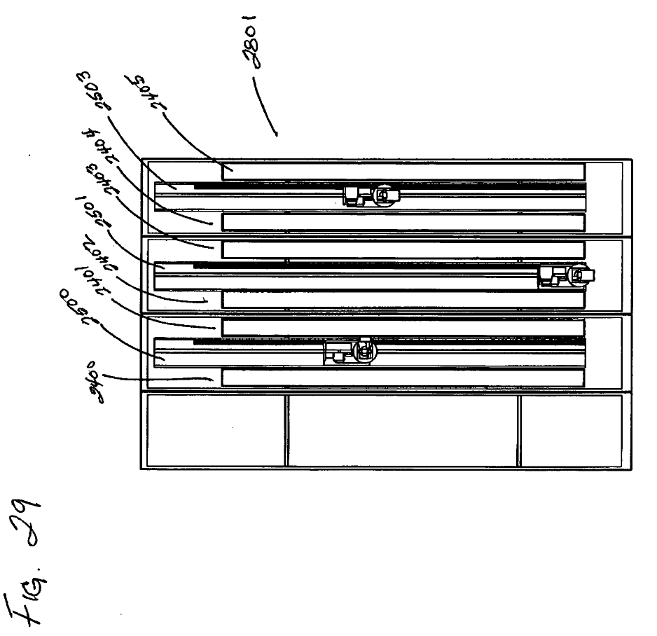

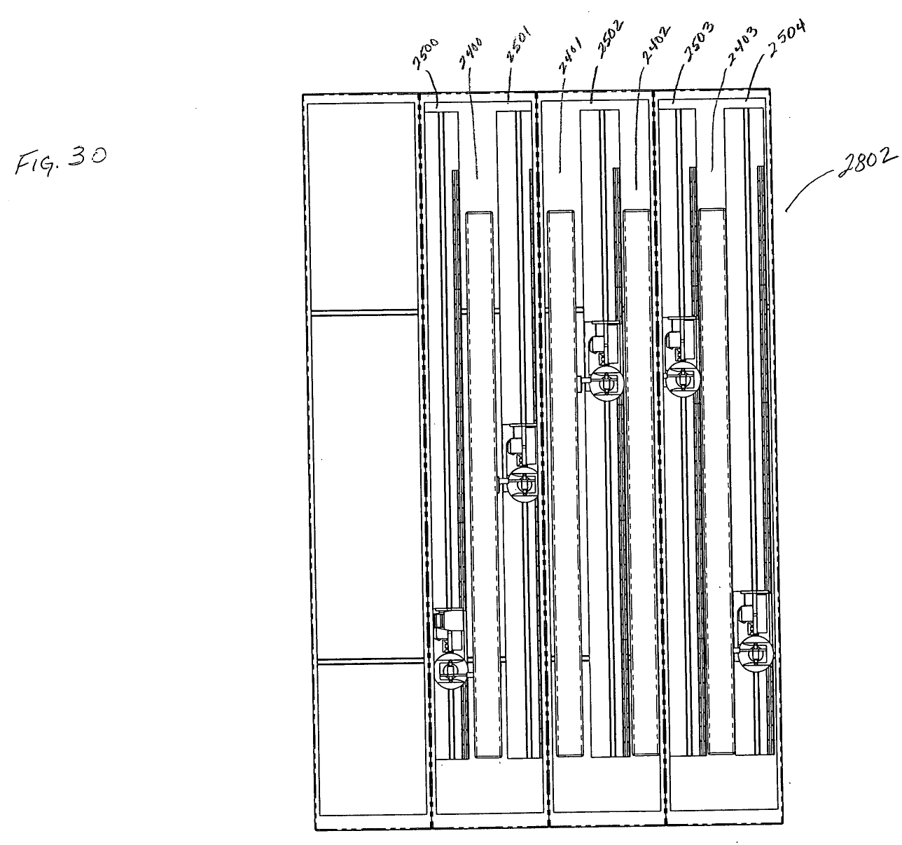

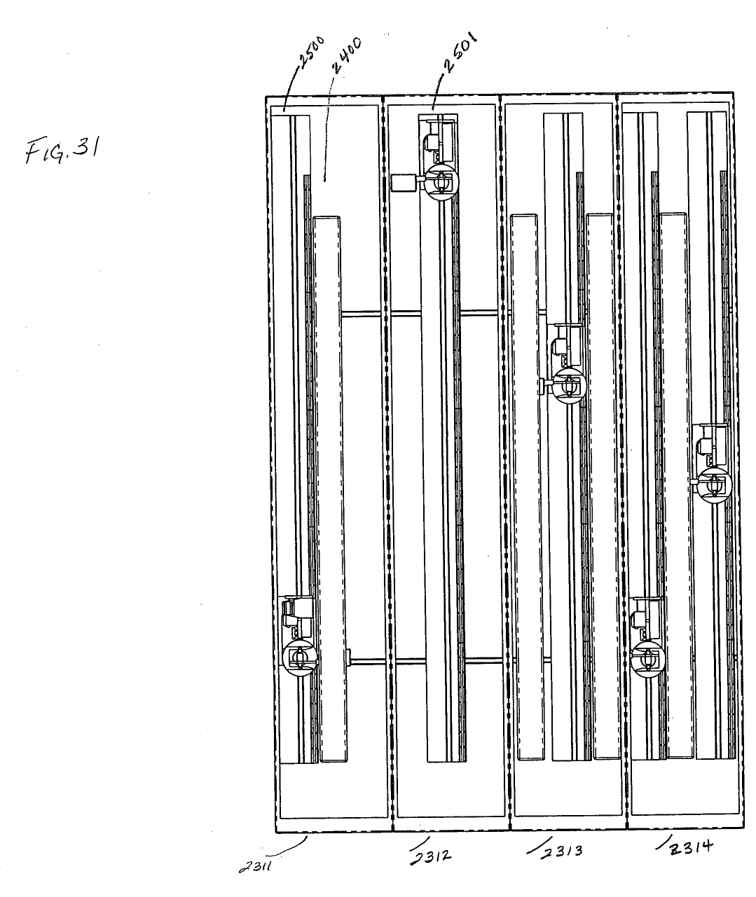

In one embodiment a modular environmental incubator comprises one or more enclosures, one or more shelves, one or more robots, one or more plate access doors for receiving and handing-off micro-plates, optionally, a means for micro-plate conveyance, a maintenance space between a primary access door, not shown in FIG. 26, and the exterior of the environmental enclosure, and various environmental control apparatus, not shown, as described previously; access to the exterior is provided by an additional door, not shown, in the maintenance space which may be at a different environmental condition from the one maintained for the portion of the enclosure containing the shelves. FIG. 26 is a schematic view of a modular incubator enclosure configured with two robots 2500 and 2501 separated by shelf 2400. Note cross-tracks 2321 and 2320. In FIG. 27, two shelves 2400 and 2401 are separated by an aisle in which robot 2500 traverses, accessing plates 2450 positioned on either shelf.

In one embodiment one or more modular environmental incubators may be coupled together to form incubator or library 2800 as shown in FIG. 28A; a library is made up of one or more modular incubators containing multiple micro-plates at a given set of environmental conditions. One or more libraries may be coupled together, operating in concert with one or more robotic systems such as a Velocity11 BioCel®. At least one of large end walls 2810 or 2811 is removable; optionally one of 2810 or 2811 may contain apparatus for environmental control; optionally some combination of end walls 2810, 2811, 2310, and 2311 contain substantially all of the required environmental control apparatus. FIG. 28B shows one embodiment with environmental control units and other control systems located on one small end wall as 2830, 2835 and 2840. Note access door 2850 enabling access to internal of incubator library 2800. Conveyance tracks 2820, 2821 and 2822 are shown at the other end of incubator enclosure 2301, 2302, 2303 and 2304; five robots 2500, others not shown, place and retrieve micro-plates 2450 on common conveyance tracks. In this embodiment conveyance tracks 2820, 2821 and 2822 are internal to incubator enclosures; alternatively they may be external. As the size of a library increases it may be economical to place it in a cleanroom specifically designed for the function; in this case a room takes on the function of an enclosure without detracting from the novelty of the invention.

FIGS. 29 and 30 are top views of alternative library configurations 2801 and 2802. In FIG. 29 library 2801 utilizes one robot to access two shelves; this configuration maximizes the storage capacity of the library foot print. In FIG. 30 is shown an alternative configuration 2802 with two robots per shelf; this configuration maximizes the speed or response time and the reliability of a library. Since a robot is the active component of the system a robot failure means that any micro-plates within its reach can not be accessed; by configuring the system such that a plate can be accessed by two independent robots the reliability of the system has been improved dramatically. In FIG. 31 the shelves and robots have been separated such that robot 2502 is accessible through doors 2311 or 2312. This extra space is provided by the use of an additional modular enclosure such as 2304 which initially has less than all of its tracks occupied. The shelves or robots are moved about either manually or with a crank or with a motor driven assembly activated by the user. When a shelf or robot is not to be moved slides 2460 and 2461 for a shelf and 2560 and 2561 for a robot are provided with latches, not shown, which prevent motion. One or more latches are used for each shelf and robot; a latch engages with a precisely positioned mating latch or hole which functions as a reference datum for accurately positioning a shelf or robot with reference to a corresponding shelf or robot. In this fashion, after a robot acquires the positional knowledge of where a shelf is located with respect to itself in a particular incubator enclosure and is subsequently moved and then returned, the robot need not be re-taught the positional information and similarly for moving a shelf and returning it to a robot.

In one embodiment involving one or more libraries or one or more environmental incubators a common conveyance means is provided external but in communication with each robot within the one or more libraries such that each robot accesses the common conveyance as shown in FIG. 32. The external common conveyance means 3210 may also have its own environmental control capability. In one embodiment a common conveyance means is one or more five axis robots located in an aisle with access to each environmental control zone plate access door associated with each shelf. In another embodiment a common conveyance means is a moving belt or track or multiple moving belts, or tracks, at different heights, where at one height a track may be moving a micro-plate from a system such as the Velocity11 BioCel® 3250 and at another height a track may be moving a micro-plate toward a system such as the Velocity11 BioCel®. In this embodiment there are at least two plate access doors for each robot in a library or incubator. In another embodiment a common conveyance means comprises a combination of moving belts or tracks, five axis robots and four axis robots. FIG. 33 is an alternative view of two libraries sharing common conveyance 3210 functioning cooperatively with system 3251.

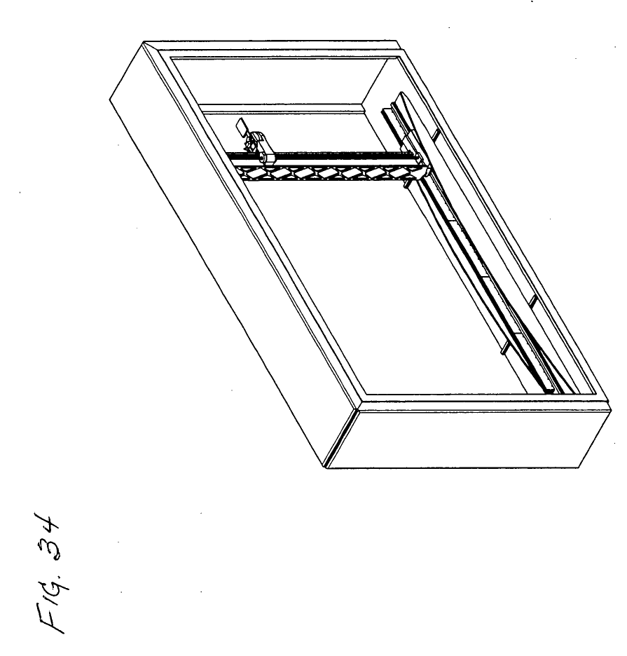

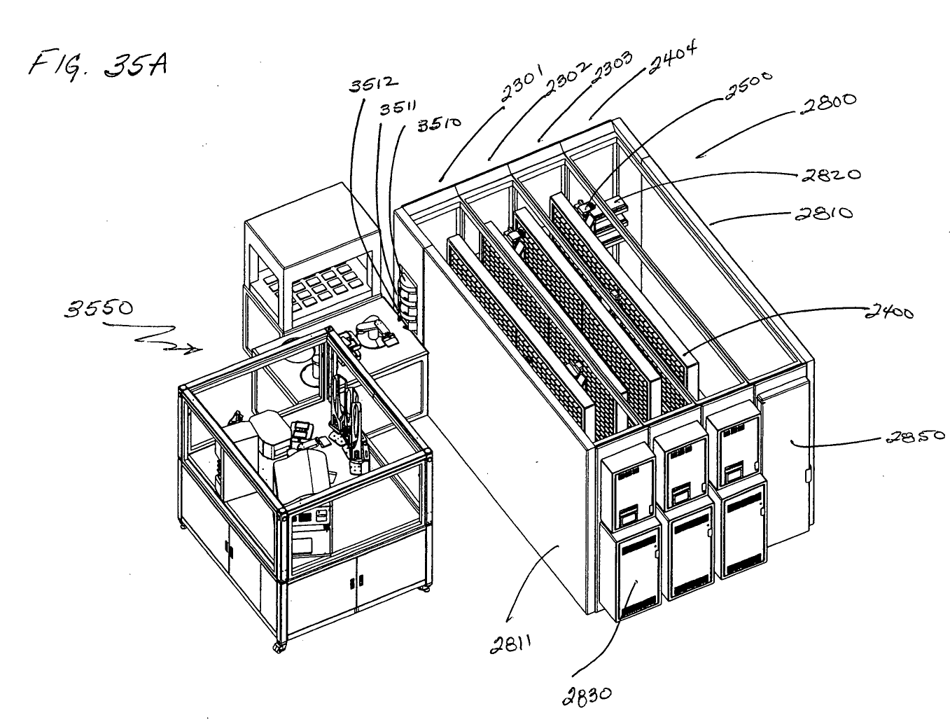

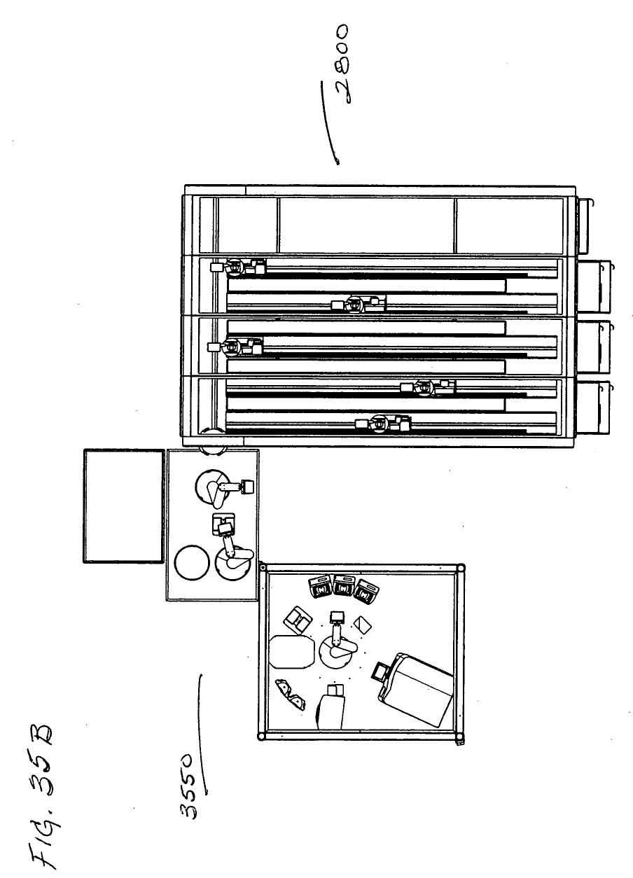

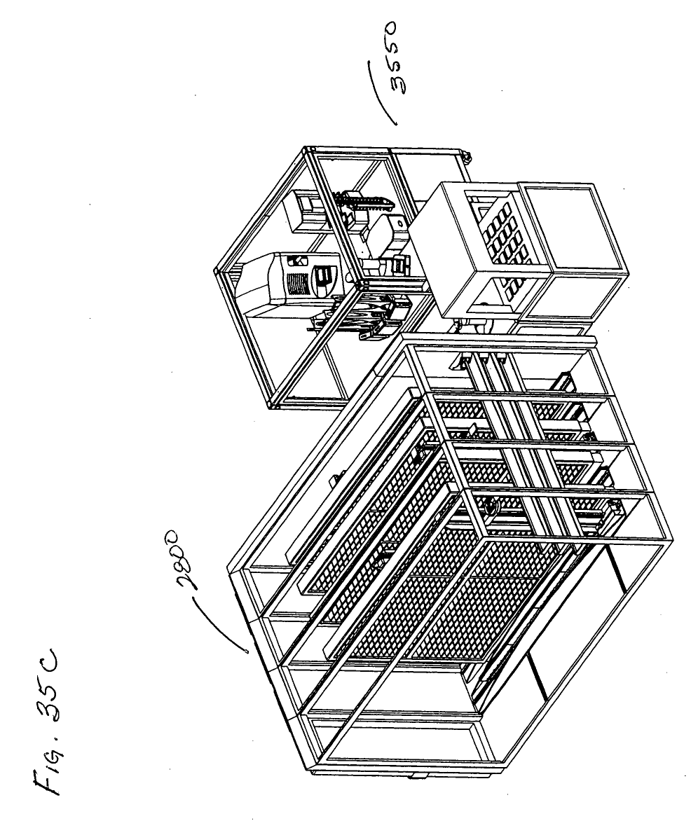

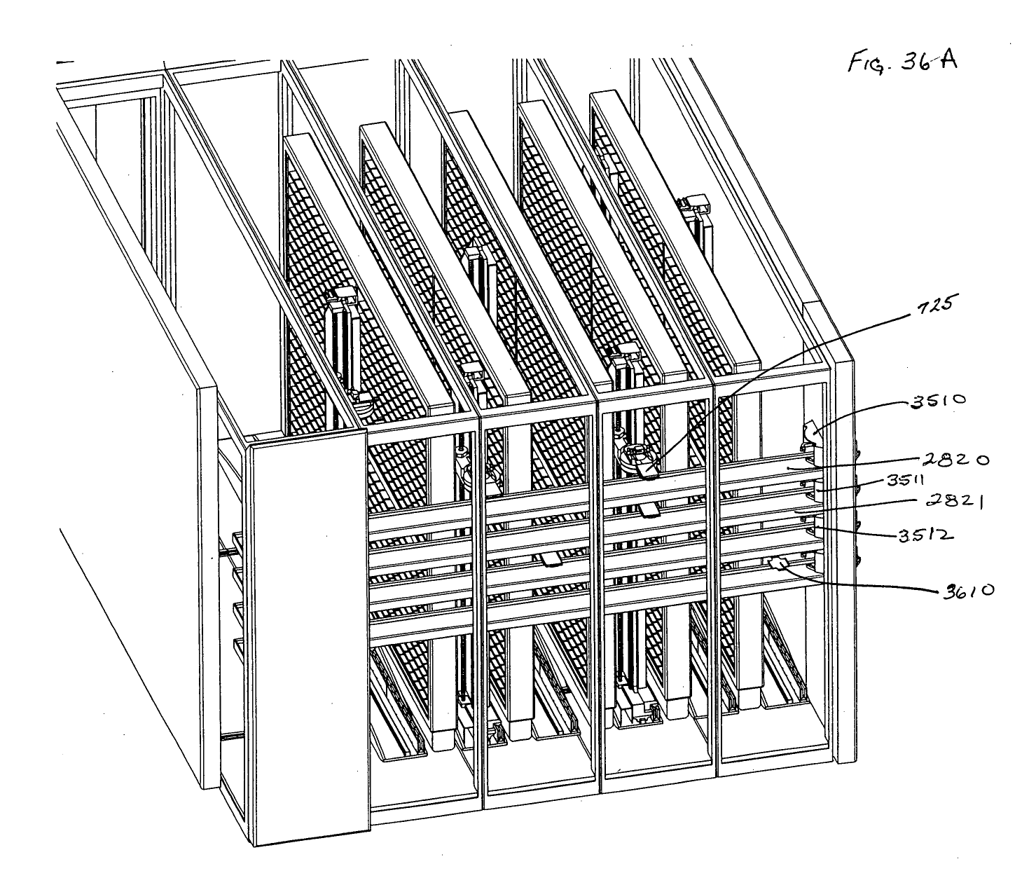

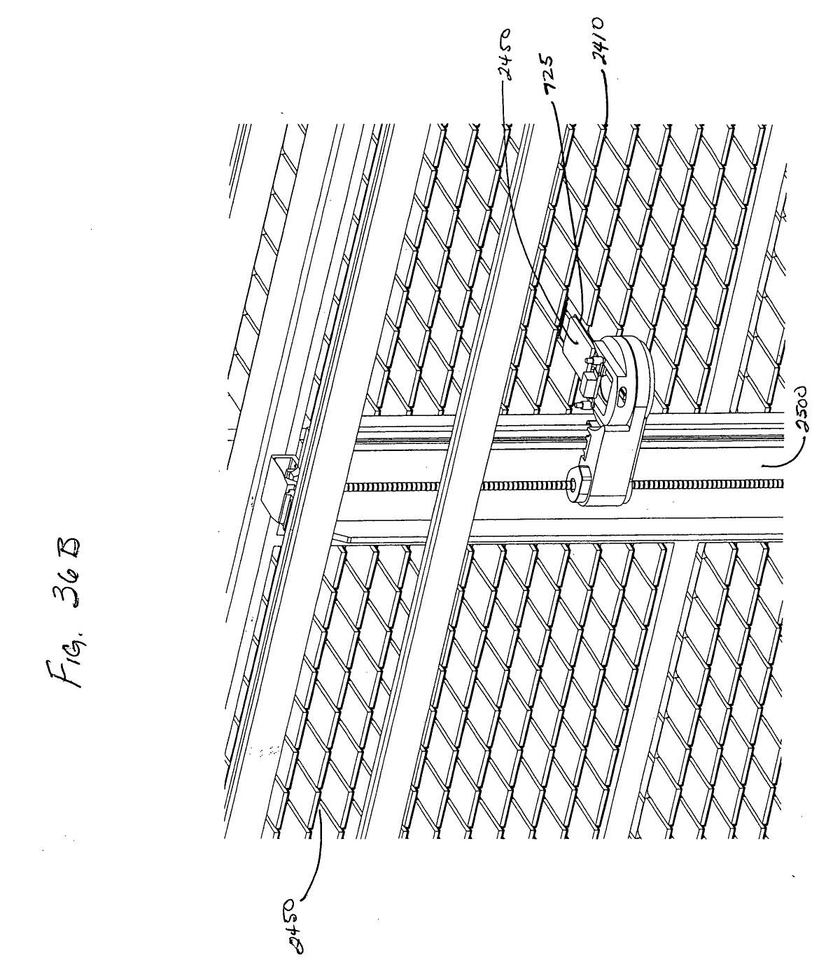

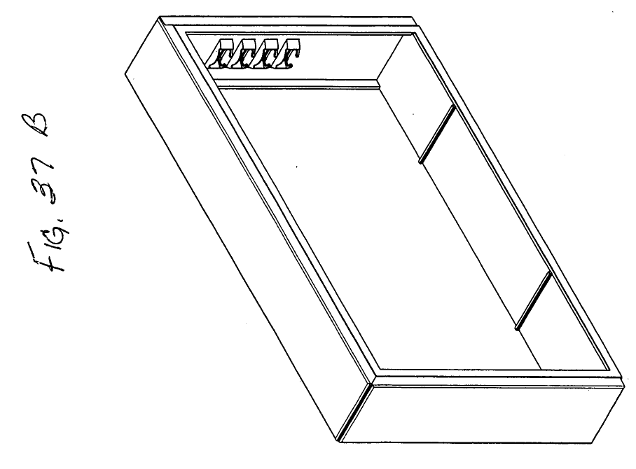

FIG. 34 is an alternative embodiment of shelves and robots configured to slide out of a small end wall of a three track environmental enclosure. FIG. 36 is one embodiment of a micro-plate access door through a small end wall.

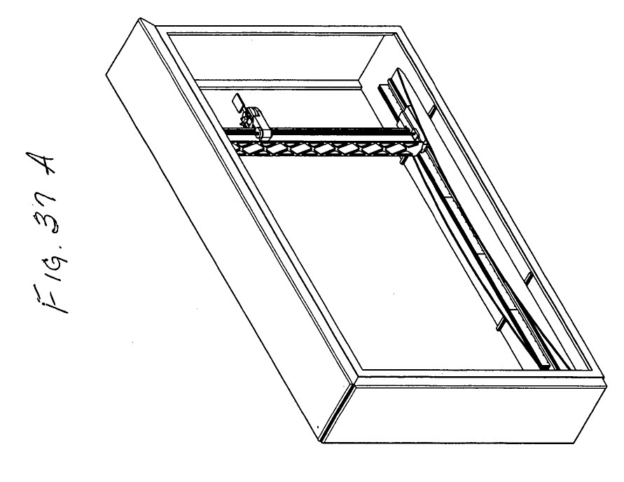

FIG. 35A shows an isometric view of an alternative embodiment of a micro-plate library comprising four environmental incubator modules 2301, four shelves 2400, five robots 2500, three environmental control units and control systems 2830, external access door 2850, three common conveyance tracks 2820, micro-plate access doors 3510, 3511 and 3512 and external robotic system 3550, such as a Velocity11 BioCel®. FIG. 35B is a top view of the same embodiment. FIG. 35C is an alternative isometric view of the same embodiment. FIG. 36A shows robot shovel 725 delivering a micro-plate to track micro-plate holder 3610 on common conveyance rack 2820 and micro-plate access door 3510 coupled to major robotic system 3550. FIG. 36B shows robot 2500 and shovel 725 delivering a micro-plate 2450 to micro-plate holder shelf 2410.

Software for controlling all of the micro-plate traffic, the placing and fetching or retrieving and setting the sequence and speed and how many robots are simultaneously fetching or returning micro-plate and other details is resident in a program in a processor and computer readable storage means in communication with an incubator and conveyance means and first or primary robotic system. The user defines the micro-plates and the order in which they should be fetched and other details related to a particular protocol in an instruction set which is processed by the program in a processor. Such a processor may be located in an incubator or library or first or primary robotic system or another more distant location; alternatively computer based processing and communication may take place over a network from an external location. Means for communicating comprise various communication protocols such as Ethernet, Device Net, RS232, 485, internet based protocols and others familiar to those knowledgeable in the art. One example of a method for operating the incubator using a first or primary robotic system such as the Velocity11 BioCel® is shown below:
a) storing an instruction set on computer readable media wherein micro-plate selection criteria are included;
b) processing the instruction set with processor in first robotic system;
c) sending a fetch command to a first robot in a first incubator wherein the fetch command contains the location coordinates of a first micro-plate;
d) fetching of first micro-plate is executed by first robot wherein first micro-plate is fetched and placed on the means for conveying;
e) instructing means for conveying to deliver first micro-plate to the first robotic system;
f) delivering first micro-plate to first robotic system by the means for conveying; and
g) repeating steps c) through f) as indicated by the instruction set for first or more robots wherein commands are sent to as many robots as required by the instruction set and resident in at least one incubator

FIG. 37A shows an isometric view of an alternative embodiment of a micro-plate access door in the embodiment the access door is in the end wall and the common conveyance is external to the incubator enclosure. FIG. 37B shows the external view of an access door; common conveyance not shown.

In one embodiment involving two or more environmental incubators micro-plates are placed in a small chamber with some environmental control capability such that the environmental change from the environmental incubator to a system such as the Velocity11 BioCel® is minimized or eliminated or controlled. In one embodiment a micro-plate is placed in a small chamber prior to exiting from its environmental incubator. In another embodiment a micro-plate is placed in a small chamber as it is placed on the common conveyance means. Alternatively, a small chamber may serve as a holding chamber for micro-plates to warm-up or cool down or in some fashion be processed prior to use or introduction to another robotic system. In one embodiment when the incubator is at a freezing temperature, −20° C. for instance, it may be necessary to thaw the material contained by a micro-plate or micro-tube prior to transferring to another step or system.

Foregoing described embodiments of the invention are provided as illustrations and descriptions. They are not intended to limit the invention to precise form described. In particular, it is contemplated that functional implementation of invention described herein may be implemented equivalently in hardware, software, firmware, and/or other available functional components or building blocks, and that networks may be wired, wireless, or a combination of wired and wireless. The described embodiments are not limited to biological processes, but also apply to micro-manufacturing and nano-manufacturing of substrates other than semiconductor wafers. Other variations and embodiments are possible in light of above teachings, and it is thus intended that the scope of invention not be limited by this Detailed Description, but rather by Claims following.

We claim:

1. A storage apparatus for holding micro-plates in a controlled environment comprising:
two drawers for storage racks;
a robot;
at least one storage rack;
an environmental control unit;
means for communicating to at least one processor;
a removable base plate; and
at least two doors;
wherein the two drawers are disposed on opposing sides of the removable base plate with the robot between the two drawers positioned to access the at least one storage rack.

2. The storage apparatus of claim 1 wherein said robot comprises a five axis robot comprising:
a X axis drive assembly comprising a spinning lead screw attached to a carriage moving on a linear bearing and a plate holder shovel attached to the carriage;
a Y axis drive assembly comprising a motor driving a lead nut on a lead screw fixed at one end;
a Z1 axis drive assembly comprising a motor driving a lead nut on a lead screw fixed at one end;
a Z2 axis drive assembly comprising a motor driving a lead nut on a lead screw fixed at one end wherein the Z2 axis drive is attached to the Z1 axis drive assembly;
a theta axis drive assembly comprising a motor driving a belt drive to rotate the shaft of the X axis at least about 180°.

3. The storage apparatus of claim 1 wherein said environmental control unit controls one or more environmental parameters with one or more means for controlling comprising:
means for producing water vapor;
means for controlling humidity;
means for controlling radiation;
means for controlling temperature;
means for controlling pressure;
means for controlling atmospheric composition;
means for controlling particle; and
means for circulating fluid;
wherein each means is programmable through said communication means.

4. The storage apparatus of claim 1 wherein said processor comprises one or more processors wherein one or more processors is external or internal to said storage apparatus.

5. The storage apparatus of claim 1 wherein said two drawers are configured for at least one storage rack each containing at least one micro-plate accessible to said robot
wherein said storage rack has machine readable symbols on a surface accessible to said robot.

6. The storage apparatus of claim 1 wherein said removable base plate comprises;
at least one connector for electrical signals to said storage apparatus;
means for mounting commonly referenced for at least two drawers and one robot; and
means for inserting into and extracting from said storage apparatus.

7. The storage apparatus of claim 1 wherein said at least two doors comprise:
at least one door which is substantially one surface of said storage apparatus; and
at least one door, sized for insertion or extraction of a micro-plate, is controlled by said processor.

8. The storage apparatus of claim 1 wherein said robot further comprises a camera attached to an axis.

9. A storage apparatus for holding plates in a controlled environment comprising:
at least four drawers for storage racks;
at least two robots;
at least two means for communicating to at least one processor;
at least two removable base plates; and
at least four doors;
wherein two drawers are disposed on opposing sides of a removable base plate with a robot between the two drawers positioned to access at least one storage rack.

10. The storage apparatus of claim 9 further comprising at least one environmental control unit.

11. The storage apparatus of claim 9 wherein said at least two robots are five axis robots comprising:
a X axis drive assembly comprising a spinning lead screw attached to a carriage moving on a linear bearing and a plate holder shovel attached to the carriage;
a Y axis drive assembly comprising a motor driving a lead nut on a lead screw fixed at one end;
a Z1 axis drive assembly comprising a motor driving a lead nut on a lead screw fixed at one end;
a Z2 axis drive assembly comprising a motor driving a lead nut on a lead screw fixed at one end wherein the Z2 axis drive is attached to the Z1 axis drive assembly;
a theta axis drive assembly comprising a motor driving a belt drive to rotate the shaft of the X axis at least about 180°.

12. The storage apparatus of claim 10 wherein said at least one environmental control unit controls one or more environmental parameters with one or more means for controlling comprising:
means for producing water vapor;
means for controlling humidity;
means for controlling radiation;
means for controlling temperature;
means for controlling pressure;
means for controlling atmospheric composition;
means for controlling particle; and
means for circulating fluid;
wherein each means is programmable through said communication means.

13. The storage apparatus of claim 9 wherein said processor comprises one or more processors wherein one or more processors is external or internal to said storage apparatus.

14. The storage apparatus of claim 9 wherein said at least four drawers are configured for at least one storage rack each containing at least one micro-plate accessible to at least one
of said at least two robots wherein said storage rack has machine readable symbols on a surface accessible to at least one of said at least two robots.

15. The storage apparatus of claim 9 wherein each of said at least two removable base plates comprises;
at least one connector for electrical signals to said storage apparatus;
means for mounting commonly referenced for at least two drawers and one robot; and
means for inserting into and extracting from said storage apparatus.

16. The storage apparatus of claim 9 wherein said at least four doors comprise:
at least two doors which each are substantially one surface of said storage apparatus; and
at least two doors, sized for insertion or extraction of a micro-plate, controlled by said processor.

17. The storage apparatus of claim 9 wherein said at least two robots further comprises a camera attached to an axis on each.

18. A storage apparatus for holding micro-plates in a controlled environment comprising:
at least one shelf with storage racks for micro-plates;
at least two robots; and
means for communicating to at least one processor;
wherein each plate is accessible by more than one robot.

19. The storage apparatus of claim 18 wherein said at least two robots are at least four axis robots comprising:
a X axis drive assembly comprising a spinning lead screw attached to a carriage moving on a linear bearing and a plate holder shovel attached to the carriage;
a Y axis drive assembly comprising a motor driving a lead nut on a lead screw fixed at one end;
a Z1 axis drive assembly comprising a motor driving a lead nut on a lead screw fixed at one end;
a theta axis drive assembly comprising a motor driving a belt drive to rotate the shaft of the X axis at least about 180°.

20. The storage apparatus of claim 18 further comprising at least one environmental control unit controlling one or more environmental parameters with one or more means for controlling comprising:
means for producing water vapor;
means for controlling humidity;
means for controlling radiation;
means for controlling temperature;
means for controlling pressure;
means for controlling atmospheric composition;
means for controlling particle; and
means for circulating fluid;
wherein each means is programmable through said communication means.

21. The storage apparatus of claim 18 wherein said processor comprises one or more processors wherein one or more processors is external or internal to said storage apparatus.

22. The storage apparatus of claim 18 wherein said at least one shelf is configured for storing at least one micro-plate accessible to at least two of said at least two robots.

23. The storage apparatus of claim 18 further comprising at least one door.

24. The storage apparatus of claim 18 wherein said at least two robots further comprises a camera attached to an axis on each.

25. The storage apparatus of claim 18 wherein said at least one shelf has machine readable symbols on surfaces accessible to two of said at least two robots.

26. A storage apparatus for holding micro-plates in a controlled environment comprising:
at least one incubator coupled with means for conveying wherein micro-plates are conveyed to or from the at least one incubator.

27. The storage apparatus of claim 26 wherein said means for conveying
comprises one or more means for conveyance chosen from a group comprising one or more moving belts, one or more moving tracks and one or more robots.

28. The storage apparatus of claim 26 further comprising:
at least one shelf for storing micro-plates in each said at least one incubator;
at least one robot in each said at least one incubator;
means for communicating to at least one processor; and
at least one door.

29. The storage apparatus of claim 28 wherein said at least one robot comprises at least four axes comprising:
a X axis drive assembly comprising a spinning lead screw attached to a carriage moving on a linear bearing and a plate holder shovel attached to the carriage;
a Y axis drive assembly comprising a motor driving a lead nut on a lead screw fixed at one end;
a Z1 axis drive assembly comprising a motor driving a lead nut on a lead screw fixed at one end;
a theta axis drive assembly comprising a motor driving a belt drive to rotate the shaft of the X axis at least about 180°.

30. The storage apparatus of claim 26 wherein said at least one incubator further comprises means for controlling one or more environmental parameters with one or more means for controlling chosen from a group comprising:
means for controlling humidity;
means for controlling radiation;
means for controlling temperature;
means for controlling pressure;
means for controlling atmospheric composition;
means for controlling particle; and
means for circulating fluid;
wherein each means is programmable through said communication means.

31. The storage apparatus of claim 28 wherein said processor comprises one or more processors wherein one or more processors is external or internal to said storage apparatus.

32. The storage apparatus of claim 28 wherein said at least one shelf is configured for for storing at least one micro-plate accessible to said at least one robot wherein said shelf may have machine readable symbols on one or more surfaces.

33. The storage apparatus of claim 26 wherein said means for conveying is coupled to a second robotic system.

34. The storage apparatus of claim 26 wherein said at least one robot further comprises a camera attached to an axis.

35. A storage apparatus for holding micro-plates in a controlled environment comprising:
at least one incubator comprising:
at least one shelf; and
at least one robot;
wherein the at least one shelf and at least one robot are mounted on cross-tracks and occupy interchangeable positions.

36. The storage apparatus of claim 35 wherein said at least one incubator is coupled to
one or more means for conveying micro-plates to or from said at least one incubator wherein means for conveying comprises one or more means for conveying chosen from a group comprising one or more moving belts, one or more moving tracks and one or more robots.

37. The storage apparatus of claim 35 further comprising means for communicating to at
least one processor wherein the at least one processor comprises one or more processors external or internal to said storage apparatus.

38. The storage apparatus of claim 35 wherein said at least one first robot comprises at least four axes comprising:
a X axis drive assembly comprising a spinning lead screw attached to a carriage moving on a linear bearing and a plate holder shovel attached to the carriage;
a Y axis drive assembly comprising a motor driving a lead nut on a lead screw fixed at one end;
a Z1 axis drive assembly comprising a motor driving a lead nut on a lead screw fixed at one end;
a theta axis drive assembly comprising a motor driving a belt drive to rotate the shaft of the X axis at least about 180°.

39. The storage apparatus of claim 35 wherein said at least one incubator further comprises means for controlling one or more environmental parameters comprising:
means for controlling humidity;
means for controlling radiation;
means for controlling temperature;
means for controlling pressure;
means for controlling atmospheric composition;
means for controlling particle; and
means for circulating fluid;
wherein each means is programmable through said communication means.

40. The storage apparatus of claim 35 wherein said at least one shelf is configured for
storing at least one micro-plate accessible to said at least one robot wherein said storage rack may have machine readable symbols on one or more surfaces accessible to said at least one first robot.

41. The storage apparatus of claim 35 further comprising at least one door.

42. The storage apparatus of claim 35 wherein said at least one robot further comprises a camera attached to an axis.

43. The storage apparatus of claim 36 wherein said means for conveying is coupled to a second robotic system.

44. The storage apparatus of claim 35 further comprising at least a second robot
cooperating in a redundant manner with said at least one robot wherein said one robot and the second robot both can access said at least one micro-plate on said at least one shelf.

45. A method for operating storage apparatus comprising at least one incubator
coupled with at least one means for conveying wherein micro-plates are conveyed to or from the at least one incubator comprising the steps of:
a) storing an instruction set on computer readable media wherein micro-plate selection criteria are included;
b) processing the instruction set with processor in first robotic system;
c) sending a fetch command to a first robot in a first incubator wherein the fetch command contains the location coordinates of a first micro-plate;
d) fetching of first micro-plate is executed by first robot wherein first micro-plate is fetched and placed on the means for conveying;
e) instructing means for conveying to deliver first micro-plate to the first robotic system;
f) delivering first micro-plate to first robotic system by the means for conveying; and
g) repeating steps c) through f) as indicated by the instruction set for first or more robots wherein commands are sent to as many robots as required by the instruction set and resident in at least one incubator
